(19) United States
(12) Patent Application Publication
(10) Pub. No.: US 2010/0204684 A1
(43) Pub. Date: Aug. 12, 2010

(54) METHODS AND SYSTEMS FOR PERFORMING NEUROINTERVENTIONAL PROCEDURES

(76) Inventors: Michi E. Garrison, Half Moon Bay, CA (US); Tony M. Chou, Hillsborough, CA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/686,202

(22) Filed: Jan. 12, 2010

Related U.S. Application Data
(60) Provisional application No. 61/144,381, filed on Jan. 13, 2009.

(51) Int. Cl. A61B 17/00

(52) U.S. Cl. 606/1

(57) ABSTRACT

A system of devices for treating cerebral artery disease or the brain includes an arterial access sheath adapted to be introduced into a common carotid, internal carotid, or vertebral artery through a penetration in the artery and receive blood from the artery. The system further includes a shunt fluidly connected to the arterial access sheath, wherein the shunt provides a pathway for blood to flow from the arterial access sheath to a return site, and a flow control assembly coupled to the shunt and adapted to regulate blood flow through the shunt. A treatment device is adapted to be introduced into the artery through the arterial access sheath and is configured to treat the cerebral artery or brain.

### CROSS-REFERENCES TO PRIORITY DOCUMENT

This application claims priority of co pending U.S. Provisional Patent Application Ser. No. 61/144,381, filed on Jan. 13, 2009. Priority of the aforementioned filing dates is hereby claimed and the disclosures of the provisional patent applications are hereby incorporated by reference in their entirety.

### BACKGROUND

The present disclosure relates generally to medical methods and devices. More particularly, the present disclosure relates to methods and systems for accessing the arterial vasculature and establishing retrograde blood flow during carotid, vertebral, and intracranial neurointerventional procedures.

Neurointerventional procedures involve the minimally invasive treatment of vascular diseases in the spine and central nervous system using interventional techniques similar to those used to treat peripheral and coronary vascular disease. The bulk of neuro-interventional procedures in the intracranial, carotid, and vertebral vasculature fall into two main categories. The first involves opening blockages in cerebral arteries caused by thrombus in the setting of acute ischemic stroke. This may involve the intra-arterial infusion of thrombolytic agents, or the recanalization of blocked arteries through mechanical, fluid infusion and/or aspiration, or active energy means.

The second category involves embolization of neurovascular pathophysiology such as aneurysms, arterial-venous fistulae or malformations, or cerebral hemorrhage. These interventions involve the placement of embolic coils, particulates, liquids, foams, and/or flow-diverting stents, to stop or redirect the flow of blood around the pathophysiology. In some procedures, balloons are used to temporarily occlude the vessels to assess the hemodynamics and patient neurologic tolerance to permanent embolization.

Other neurointerventional procedures include treatment of stenosis or vasospasm using balloon angioplasty, stent implantation, or localized drug delivery. Some neuro interventions are used in conjunction with neurosurgical operations, for example the embolization of vessels feeding a brain tumor to reduce tumor size prior to surgery, or suction decompression of large cerebral or carotid aneurysms during surgical clipping to assist the clipping procedure.

Some Exemplary Issues with Current Technology

Interventions in the intracranial vasculature often have special access challenges. Most neurointerventional procedures use a transfemoral, transbrachial, or transradial access to the carotid or vertebral artery and thence to the target intracranial artery. However, these access routes are often tortuous and may contain stenosis plaque material in the aortic arch and carotid and brachiocephalic vessel origins, presenting a risk of embolic complications during the access portion of the procedure. In addition, the cerebral and other intracranial vessels are usually much narrower than coronary or other peripheral vasculature. In recent years, interventional devices such as wires, guide catheters, stents and balloon catheters, have all been scaled down and been made more flexible to better perform in the neurovascular anatomy. However, many neurointerventional procedures remain either more difficult or impossible because of device access challenges. In time-critical procedures, for example in the treatment of acute ischemic stroke where “time is brain,” these extra difficulties may have a significant clinical impact.

In some instances, a direct puncture of the carotid or vertebral artery is attempted, to circumvent aortic arch disease and/or vessel tortuosity. Though these approaches have allowed a faster and more direct route to the cerebral vasculature they are not widely used. Transcervical percutaneous puncture sites have been associated with bleeding complications. Transcervical hematomas are particularly dangerous due to the difficulty in achieving good manual compression, as well as the risk of airway closure. Surgical cut-downs to the carotid or vertebral artery enable a suture-based vascular surgical closure of the arterial access site, to reduce this risk. However, surgical cut-downs require a higher level of skill and invasiveness and restrict the procedure to surgically trained interventionalists.

Another challenge of neurointerventional procedures is the risk of emboli. In any vascular diagnostic or interventional procedure, there is a risk of embolic particles being generated. This is more so in interventions involved stenotic or thrombotic disease, which constitute the bulk of interventional procedures. Even in procedures such as diagnostic angiography or coil placement for cerebral aneurysm filling, however, studies have shown evidence of emboli. In cerebral interventions, embolic complications have severe neurologic consequences and can result in temporary or permanent neurologic deficits or even death.

To reduce this risk, embolic protection devices and systems are commonly used in carotid artery interventions. These devices are designed to prevent embolic material from entering the cerebral vasculature during balloon angioplasty and/or stenting of carotid artery stenoses. Types of devices include intravascular filters, and reverse flow or static flow systems. Unfortunately, because of the access challenges as well as the need for rapid intervention in some cases, embolic protection systems are not used in interventions in the cerebral circulation.

### SUMMARY

Disclosed are methods and devices that allow safe, rapid and relatively short and straight access to the intracranial arteries for the introduction of interventional devices. Also disclosed are means to securely close the access site to avoid the potentially devastating consequences of a transcervical hematoma. The methods and devices include a vascular access with retrograde flow system that can be used safely and rapidly in these neurointerventional procedures. The system offers the user a degree of flow control so as to address the specific hemodynamic requirements of the cerebral vasculature.

In one aspect, there is disclosed a system of devices for treating cerebral artery disease or the brain that includes an arterial access sheath adapted to be introduced into a common carotid, internal carotid, or vertebral artery through a penetration in the artery and receive blood from the artery. The system further includes a shunt fluidly connected to the arterial access sheath, wherein the shunt provides a pathway for blood to flow from the arterial access sheath to a return site, and a flow control assembly coupled to the shunt and adapted to regulate blood flow through the shunt. A treatment device is adapted to be introduced into the artery through the arterial access sheath and is configured to treat the cerebral artery or brain.

In another aspect, there is disclosed a system of devices for treating cerebral artery disease or the brain, comprising: an arterial access sheath adapted to be introduced into a common carotid, internal carotid, or vertebral artery through a penetration in the artery and receive blood from the artery; a treatment device adapted to be introduced into the artery through the arterial access sheath and configured to treat the cerebral artery or the brain; and a closure device adapted to provide a closure force to the penetration.

In another aspect, there is disclosed a method for treatment of cerebral artery disease or the brain, comprising: forming a penetration in a wall of a common carotid, internal carotid, or vertebral artery; positioning an arterial access sheath through the penetration into the artery; occluding at least one of the common or internal carotid artery or vertebral or basilar artery; and treating the cerebral artery or the brain using a treatment device.

In another aspect, there is disclosed a method for treatment of cerebral artery disease or the brain, comprising: forming a percutaneous penetration in a wall of a common carotid, internal carotid, or vertebral artery; applying a closure device at a site of penetration before placement of an arterial access sheath; positioning an arterial access sheath through the penetration into the artery; treating the cerebral artery or the brain using a treatment device; and closing the access site with the closure device

In another aspect, there is disclosed a method for treatment of cerebral artery disease or the brain, comprising: forming a penetration in a wall of a common carotid, internal carotid or vertebral artery; positioning an arterial access sheath through the penetration; inserting a treatment device through the arterial access sheath into the common carotid or vertebral artery; positioning at least a portion of the treatment device in the cerebral artery; treating the cerebral artery or the brain using the treatment device.

Other features and advantages should be apparent from the following description of various embodiments, which illustrate, by way of example, the principles of the invention.

### BRIEF DESCRIPTION OF THE DRAWINGS

FIG. 1 schematically depicts normal, antegrade cerebral circulation.

FIG. 2A depicts the blood flow circulation after retrograde flow has been established using the retrograde flow system described herein.

FIG. 2B depicts the blood flow circulation after retrograde flow has been established using the retrograde flow system described herein.

FIG. 3 shows the cerebral vasculature with a mechanical intervention device inserted through an exemplary arterial access device.

FIG. 4 shows an exemplary embodiment of a vascular access and reverse flow system that can be used to establish retrograde flow during treatment of an aneurysm.

FIG. 5 shows a side view of an exemplary embodiment of an intervention device.

FIG. 6 shows an enlarged view of a distal region of the exemplary intervention device.

FIGS. 7 and 8 show alternate embodiments of the intervention device.

FIGS. 9A and 9B show enlarged views of a distal region of an exemplary intervention device positioned near an aneurysm.

FIG. 10A illustrates an arterial access device useful in the methods and systems of the present disclosure.

FIG. 10B illustrates an additional arterial access device construction with a reduced diameter distal end.

FIGS. 11A and 11B illustrate a tube useful with the sheath of FIG. 10A.

FIG. 12A illustrates an additional arterial access device construction with an expandable occlusion element.

FIG. 12B illustrates an additional arterial access device construction with an expandable occlusion element and a reduced diameter distal end.

FIG. 13 illustrates a first embodiment of a venous return device useful in the methods and systems of the present disclosure.

FIG. 14 illustrates an alternative venous return device useful in the methods and systems of the present disclosure.

FIG. 15 shows an example of the reverse flow system with a schematic representation of the flow control assembly.

FIG. 16A-16D, FIGS. 17A-17D, FIGS. 18A and 18B, FIGS. 19A-19D, and FIGS. 20A and 20B, illustrate different embodiments of a variable flow resistance component useful in the methods and systems of the present disclosure.

FIGS. 21A-21B, FIGS. 22A-22B, FIGS. 23A-23D, and FIGS. 24A-24B illustrate further embodiments of a variable flow resistance system useful in the methods and systems of the present disclosure.

### DETAILED DESCRIPTION

Disclosed are methods and devices that allow safe, rapid and relatively short and straight access to the intracranial arteries for the introduction of interventional devices. Also disclosed are means to securely close the access site to avoid the potentially devastating consequences of a transcervical hematoma. The methods and devices include a vascular access with retrograde flow system that can be used safely and rapidly in these neurointerventional procedures. The system offers the user a degree of flow control so as to address the specific hemodynamic requirements of the cerebral vasculature.

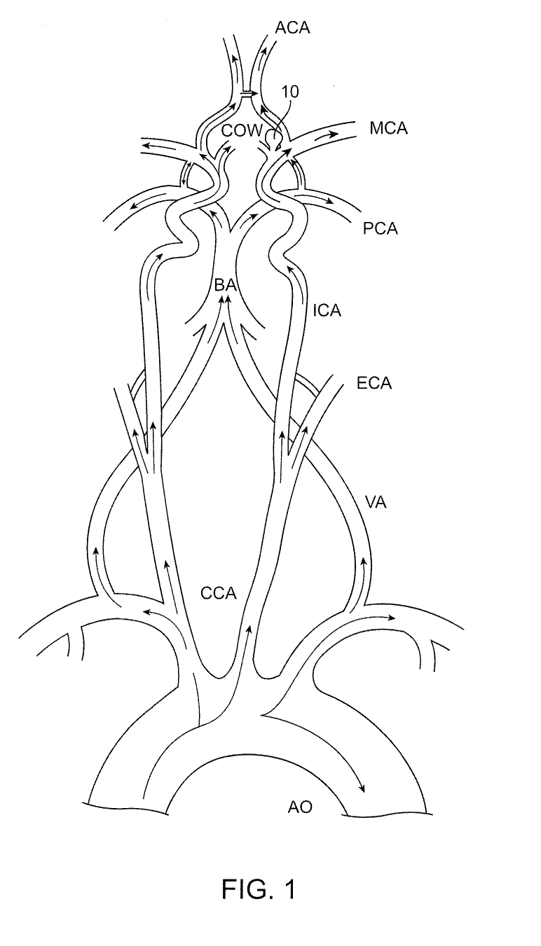

FIG. 1 schematically depicts normal, antegrade cerebral circulation. The cerebral circulation is supplied from the right and left common carotid arteries CCA, each of which branch into an external carotid artery ECA and an internal carotid artery ICA. The internal carotid artery branches into a middle cerebral artery MCA and anterior cerebral artery ACA. The cerebral arteries are also supplied by the right and left vertebral arteries VA which combine to form the basilar artery BA. The basilar artery BA branches into the right and left posterial cerebral arteries PCA. Communicating arteries connect the posterior, middle, and anterior cerebral arteries to form a circular connection called the Circle of Willis COW.

An aneurysm 10 is located for example in the bifurcation of the internal carotid artery ICA. An aneurysm is a localized, blood-filled bulge of a blood vessel caused by disease or weakening of the vessel wall. As the size of an aneurysm increases, there is an increased risk of rupture, which can result in severe hemorrhage or other complications including sudden death.

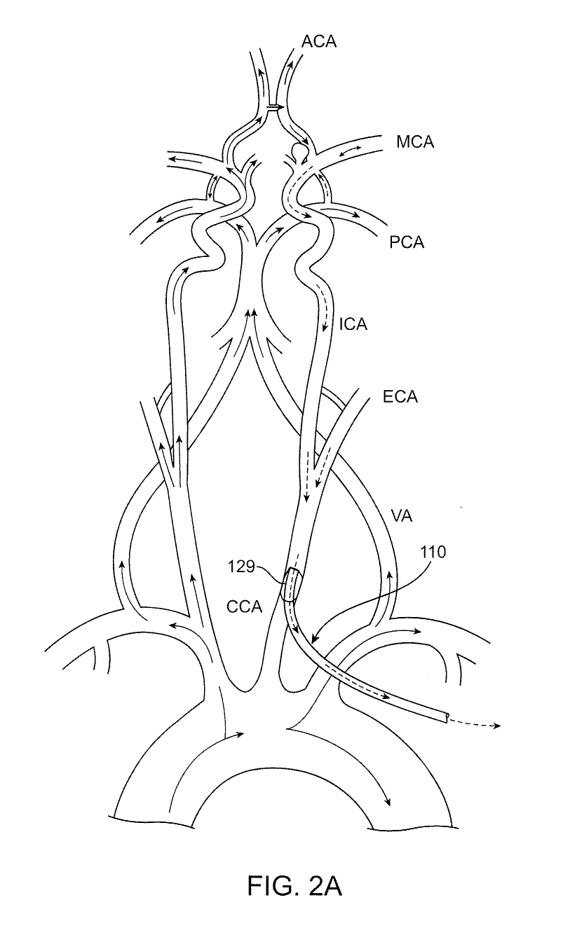

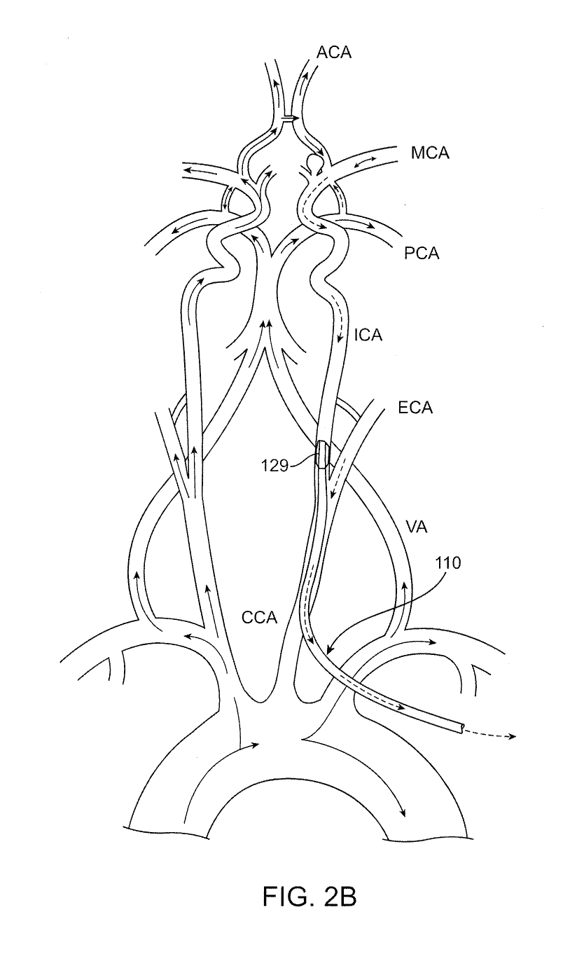

Pursuant to use of methods and systems described herein, a neurointerventional treatment method includes obtaining vascular access to the intracranial arteries and establishing retrograde flow in at least a portion of the cerebral circulation in order to safely perform an intervention with respect to the aneurysm 10. Retrograde flow is sometimes referred to as reverse flow. FIG. 2A depicts the blood flow circulation after retrograde flow has been established using the retrograde flow system described herein. The system includes an arterial access device 110 that enters the common carotid artery CCA via a penetration in the CCA to provide access to the cerebral vasculature. The arterial access device may also enter another artery, such as the internal carotid artery or vertebral artery via a penetration in such artery. An expandable occlusion element 129 on the arterial access device 110 can be used to occlude an artery in the cerebral vasculature and establish retrograde flow, as described more fully below. Various arteries may be occluded including, for example, the common carotid artery, internal carotid artery, basilar artery, and/or vertebral artery. FIG. 2A shows an example of the common carotid artery being occluded while FIG. 2B shows an example of the internal carotid artery being occluded. Exemplary embodiments of the system and its components are described in detail below.

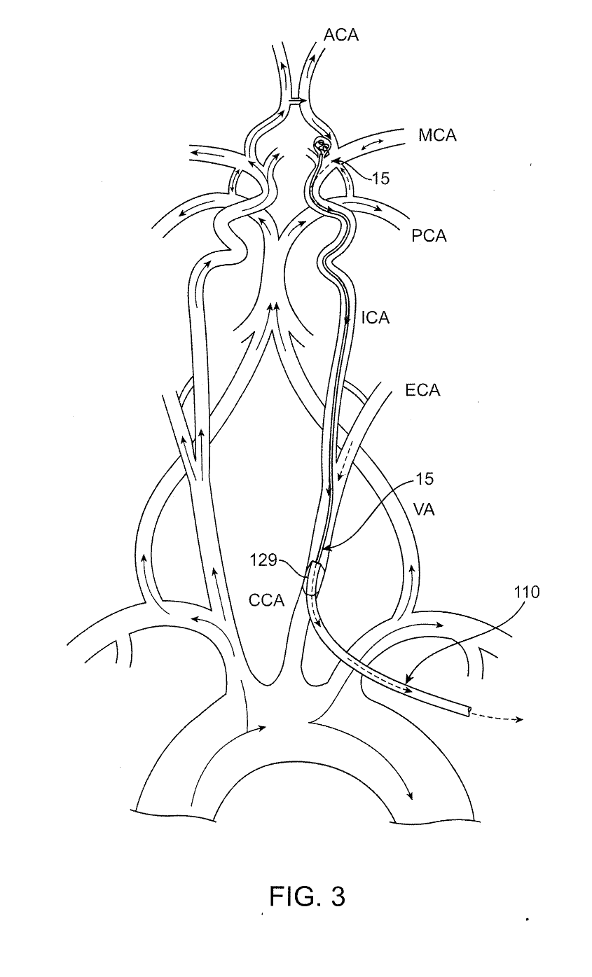

FIG. 3 shows the cerebral vasculature with a mechanical intervention device 15 (also referred to as a treatment device) inserted through the arterial access device 110. The intervention device 15 includes an elongate catheter that can be advanced through the arterial access device 110 to the location of the aneurysm 10. The intervention device 15 has a distal region that includes an element that is adapted to interact with and treat the aneurysm 10, as described more fully below. Various embodiments of the intervention device 15 are described below.

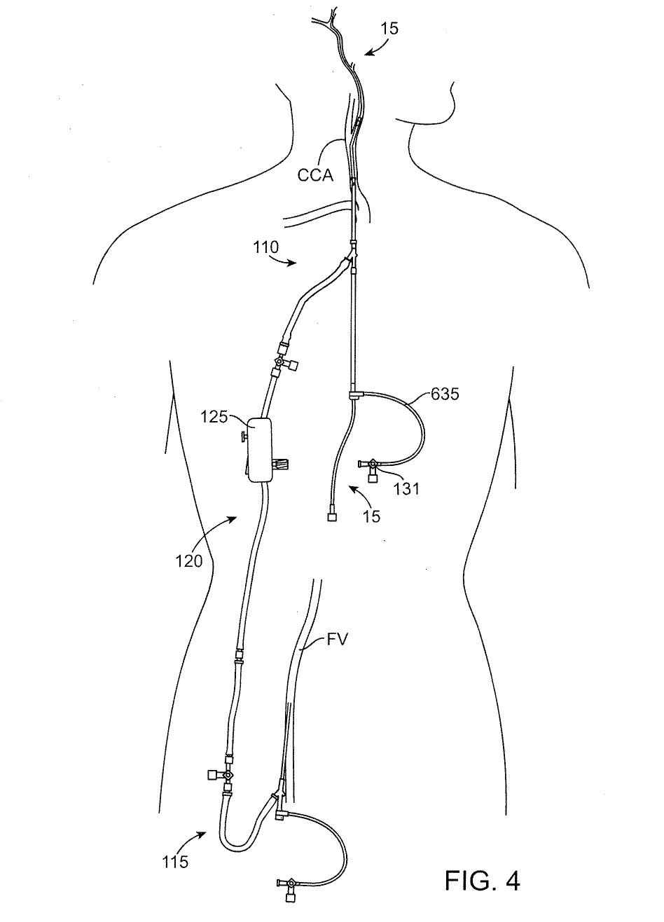

FIG. 4 shows an exemplary embodiment of a vascular access and reverse flow system 100 that can be used to establish retrograde flow during treatment of the aneurysm 10. The system 100 includes the arterial access device 110, a venous return device 115, and a shunt 120 that provides a passageway for retrograde flow from the arterial access device 110 to the venous return device 115. A flow control assembly 125 interacts with the shunt 120. The flow control assembly 125 is adapted to regulate and/or monitor the retrograde flow through the shunt 120, as described in more detail below. The flow control assembly 125 interacts with the flow pathway through the shunt 120, either external to the flow path, inside the flow path, or both.

In an embodiment, the arterial access device 110 at least partially inserts into the common carotid artery CCA and the venous return device 115 at least partially inserts into a venous return site, such as the femoral vein or internal jugular vein, as described in more detail below. The venous return device 115 can be inserted into the femoral vein FV via a percutaneous puncture in the groin. The arterial access device 110 and the venous return device 115 couple to opposite ends of the shunt 120 at connectors. The distal end of the arterial access device 110 with the occlusion element 129 may be positioned in the ICA. Alternately, in some circumstances where the ICA access is extremely tortuous, it may be preferable to position the occlusion element more proximally in the common carotid artery. When flow through the internal carotid artery is blocked (using the occlusion element 129), the natural pressure gradient between the internal carotid artery and the venous system causes blood to flow in a retrograde or reverse direction from the cerebral vasculature through the internal carotid artery and through the shunt 120 into the venous system. The flow control assembly 125 modulates, augments, assists, monitors, and/or otherwise regulates the retrograde blood flow.

The intervention device 15 is deployed to the site of the cerebral aneurysm 10 through the arterial access device and via the internal carotid artery. A distal region of the intervention device 15 is positioned in interaction with the aneurysm 10, as described more fully below. A proximal region of the intervention device 15 protrudes from an access port in the arterial access device 110. As discussed, an exemplary manner in which the intervention device 15 treats the aneurysm 10 is described in detail below.

As discussed, the arterial access device 110 provides access to the anterior and middle cerebral arteries via the common carotid artery CCA using a transcervical approach. Transcervical access provides a short length and non-tortuous pathway from the vascular access point to the target treatment site thereby easing the time and difficulty of the procedure, compared for example to a transfemoral approach. Additionally, this access route reduces the risk of emboli generation from navigation of diseased, angulated, or tortuous aortic arch or common carotid artery anatomy. In another embodiment, the arterial access device provides access to the basilar artery BA or posterior cerebral arteries PCA via a cut down incision to in the vertebral artery or a percutaneous puncture of the vertebral artery.

In an embodiment, transcervical access to the common carotid artery is achieved percutaneously via an incision or puncture in the skin through which the arterial access device 110 is inserted. If an incision is used, then the incision can be about 0.5 cm in length. An occlusion element 129, such as an expandable balloon, can be used to occlude the internal carotid artery ICA or the common carotid artery CCA at a location proximal of the distal tip of the arterial access device 110. The occlusion element 129 can be located on the arterial access device 110 or it can be located on a separate device. In an alternate embodiment, the arterial access device 110 accesses the common carotid artery CCA via a direct surgical transcervical approach. In the surgical approach, the common carotid artery can be occluded using a tourniquet.

In another embodiment, the arterial access device 110 accesses the common carotid artery CCA via a transcervical approach while the venous return device 115 access a venous return site other than the femoral vein, such as the internal jugular vein. In another embodiment, the system provides retrograde flow from the carotid artery to an external receptacle rather than to a venous return site. The arterial access device 110 connects to the receptacle via the shunt 120, which communicates with the flow control assembly 125. The retrograde flow of blood is collected in the receptacle 130. If desired, the blood could be filtered and subsequently returned to the patient. The pressure of the receptacle 130 could be set at zero pressure (atmospheric pressure) or even lower, causing the blood to flow in a reverse direction from the cerebral vasculature to the receptacle 130. Optionally, to achieve or enhance reverse flow from the internal carotid artery, flow from the external carotid artery can be blocked, typically by deploying a balloon or other occlusion element in the external carotid artery just above the bifurcation with the internal carotid artery.

In another embodiment, reverse flow may be replaced or augmented by application of an aspiration source to a port 131 (such as a stopcock) that communicates with the flow shunt 120. Examples of an aspiration source include a syringe, pump, or the like. Alternately, the system may include an active pump as part of the flow control assembly 125, with controls for pump flow rate and/or flow monitoring included in the assembly.

In yet another embodiment, the system may be used to deliver intra-arterial therapeutic agents such as thrombolytic agents under static flow or under intermittent or continuous reverse flow conditions. For example, thrombolytic therapy may be infused to a treatment site through the arterial access device 110 via a flush line 635. In another embodiment, the system may be used to deliver a pharmacologic agent or agents via a micro catheter which is inserted into the arterial access device 110. The distal end of the micro catheter is delivered to the treatment site to infuse the agent. Static or reverse flow may be initiated during delivery of the micro catheter and/or the therapeutic agent to minimize the risk of embolic particles entering the brain during this procedure

In yet another embodiment, the system may be used to perform an intracranial or cerebral angiogram under static flow or under intermittent or continuous reverse flow conditions. For example, a diagnostic catheter is inserted in to the arterial access device 110 of the reverse flow system. The diagnostic catheter is used to delivery contrast media to the relevant arterial sites. Static or reverse flow may be initiated during delivery of the diagnostic catheter and/or during delivery of the contrast media, to minimize the risk of embolic particles during this procedure.

In yet another embodiment, the system may be used to treat an intracranial stenosis under angiogram under static flow or under intermittent or continuous reverse flow conditions. For example an angioplasty balloon catheter is inserted into the arterial access device 110. The balloon catheter is positioned at the site of the stenosis and the balloon is treated to dilate the stenosis. Alternately, an intracranial stent is inserted into the arterial access device 110 and deployed at the site of the stenosis. Static or reverse flow may be initiated during delivery of the balloon or stent delivery catheter to minimize the risk of embolic particles during this procedure.

It can be appreciated that other neurointerventions can be performed with this system under static flow or under intermittent or continuous reverse flow conditions to minimize the risk of embolic complications during these procedures.

### Exemplary Embodiments of Intervention Device

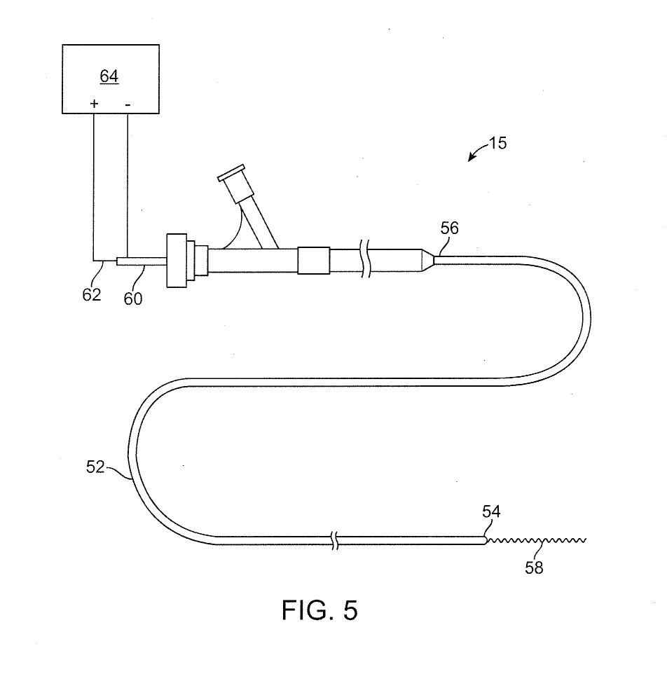

FIG. 5 shows a side view of an exemplary embodiment of the intervention device 15. The intervention device includes an endovascular implant that is adapted to stabilize and at least partially fill a vascular aneurysm. In an embodiment, the intervention device is used pursuant to a procedure that includes the steps of filling the selected aneurysm with an endovascular implant such as a coil. The endovascular implant is configured to at least partially fill the aneurysm. U.S. Pat. No. 5,522,836 and U.S. Pat. No. 5,749,894 (both incorporated by reference herein) describe exemplary systems and methods for filling an aneurysm. It should be appreciated that other types of endovascular implants or materials can be used to treat the aneurysm and that the following description is exemplary and non-limiting.

With reference still to FIG. 5, the device 15 includes an elongate catheter body 52 which has a distal end 54 and a proximal end 56. The catheter body 52 includes least one lumen passing between the distal end 54 and the proximal end 56. Passing through the lumen of catheter body 52 are a collection of components. The components can include a detachable coil 58 that protrudes from the distal end 54 of the catheter body 52 as the coil 58 is deployed. A pusher 60 may be used to push the detachable coil 58 out of the distal end 54 of the catheter body 52, as described below. When used, a core wire 62 extends from the catheter body's distal end through pusher 60 and into the center of the detachable coil 58.

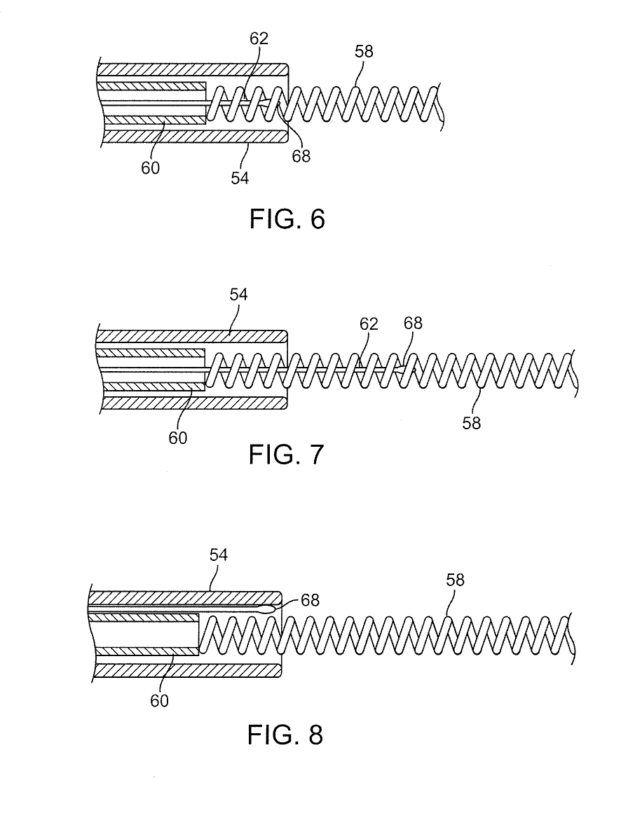

A circuit for electrolytically detaching a desired portion of the detachable coil 58 passes through a conductive path defined by the pusher 60 and the core wire 62. A small gap may be located between the detachable coil 108 and a distal electrode 68 (FIG. 6) on the distal region of the core wire 62. A power supply 64 is coupled to the core wire 62. The core wire 62 may be covered with an insulating material such as polyfluorocarbons (e.g., Teflon), polyurethane, polyethylene, polypropylene, or other suitable polymeric material. The electrode 68, which is discussed in more detail below, is not covered with the electrical insulator and is of a material that should not dissolve in the blood upon imposition of the voltage. In an embodiment, the core wire 62 in the region of its distal section is of a metal which is more noble than that found in the detachable coil 58. The core wire 62 can be 10-50 mils. in diameter and is of stainless steel or the like. The core wire 62 and the entire intervention device 15, is typically between 50 and 300 cm. in length. The length of the intervention device 15 is chosen based upon the use to which the device is to be placed.

FIG. 6 shows an enlarged view of a distal region of the exemplary intervention device 15 in which the core wire 62 is immobile with respect to the distal end 54 of the catheter body. As was noted above, the core wire 62 is coated with an insulator up to the region of the distal electrode 68. The distal electrode 68 is left uncoated so to allow an electrical path to form through the liquid surrounding it to the detachable coil 58. This variation of the device operates in the following fashion. The pusher 60 pushes the detachable coil 58 through the catheter body 52 until the desired length of the detachable coil 58 has emanated through the distal end of the catheter lumen. The immobile core wire 62 does not move with respect to catheter body 52. This variation permits the attending physician to understand that the length of the detachable coil 58 which extends beyond the tip of the catheter is the length of detachable coil 58 which will be left at the selected treatment site, such as at the aneurysm.

The electrode 68 at the tip of immobile core wire 62 should, at once, be both open to the fluid in the vasculature so to allow the electrolysis to take place but also not be allowed to contact the interior of the coil 58 lest a direct short take place. A shroud or protector is desirably placed over the electrode 68. The core wire 62 itself is insulated proximally of the electrode 68. Inherently slippery polymers as polyfluorocarbons (such as PTFE, FEP), polysulfones or the like are desirable as such coatings.

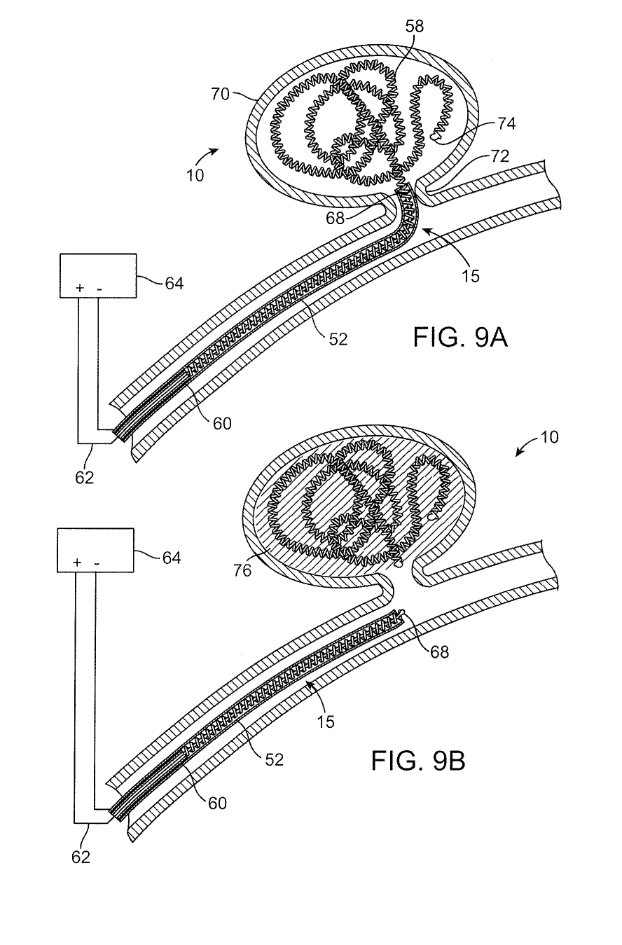

FIG. 7 shows another variation of the intervention device 15. In this embodiment, the detachable coil 58 may be electrolytically severed outside of the catheter body distal tip (104). This is accomplished by use of a movable core wire 62. The core wire 62 may be axially moved within the inner lumen of coil 58 and with respect to the distal end 54 of the catheter body 52. This variation allows the attending physician to trim the length of detachable coil 58 at some determinable point outside of the catheter body. This may be desirable, for instance, when occluding an aneurysm. In this way, the distal end 54 of the catheter body 52 is positioned near the opening of the aneurysm, the proper length of detachable coil 58 is then placed through the mouth of the aneurysm into the sac, and the electrode 68 on the core wire 62 is then inserted just into the aneurysm so that during electrolytic dissolution of a small section of the coil, the dissolution takes place within the aneurysm sac beyond the aneurysm neck. This prevents any small sections of coil remaining out in the artery to form other non-desired emboli.

FIG. 8 shows another variation in which no core wire is used. The device employs a pusher 60 and a detachable coil 58. However, in lieu of the electrode found interior to the detachable coil 58 as in FIGS. 6 and 7, the electrode 68 in this variation is located on the interior of the catheter distal section 54. This configuration has many of the same benefits as does the variation shown in FIG. 6 in that the attending physician is cognizant of the amount of coil to be left at the desired occluded site because that amount of coil equals that amount seen emanating from the distal end 54 of the catheter body.

The catheter body in this variation has included within its wall (or otherwise provided for), a conductor which extends from the proximal end of the catheter to the electrode 68. It should be apparent that the pusher 60 completes the circuit through the detachable coil 58 either by inclusion of a conductive wire in the wall of the pusher 60 or by a discrete wire passing through the lumen of the pusher. In the variations shown in FIGS. 6 and 7, it is more desirable to place the conductor in the wall of the pusher since in that way, the movement of core wire 62 is not impeded. In the variation shown in FIG. 8, the conductor associated with the proximal end of the detachable coil 58 may either be placed within the wall of the pusher 60 or through the lumen found in the midsection. Indeed, in certain short catheter assemblies may be completely metallic. Other means of conducting electricity to the proximal end of the detachable coil 58 can also be used.

As was the case in the variation found in FIG. 7, the electrode 68 may be provided with a protector or shroud to allow the contact of the metallic electrode 68 with blood but not to allow the electrode to contact the interior of coil 58. Also as was the case with immobile core wire 62, the core wire 62 is insulated proximally of the metallic tip 68 such as with a lubricious polymer.

The endovascular implant shown in each of the drawings above is shown to be a coil 58. The endovascular implant may be a coil or it may be some other embolic material, such as cyanoacrylates, ethylene-vinyl alcohol copolymer mixtures, Ethibloc, ethanol, estrogen, poly(vinyl acetate), cellulose acetate polymer, poly(vinyl alcohol), gelatin sponges, microfibrillar collagen, surgical silk sutures, detachable balloons, or other embolic agents which are delivered through a microcatheter.

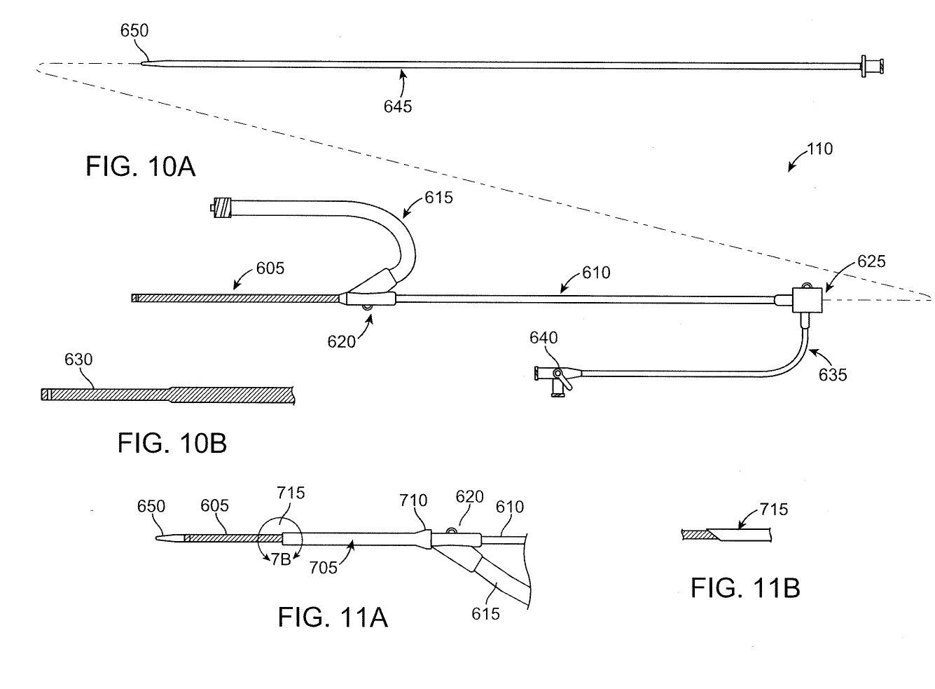

An exemplary procedure for using the device 15 to treat the aneurysm 10 in combination with the vascular access and reverse flow system is now described. The arterial access device 110 is introduced into the common carotid artery CCA of the patient and positioned in the distal common carotid artery or internal carotid artery, as shown in FIG. 4. The intervention device 15 is then advanced through the arterial access device 110 into the carotid artery. Before advancing the intervention device 15 further, the occlusion element 129 on the arterial access device 110 may be expanded to reduce or even stop antegrade flow through the vessel. Stopping flow in the vessel may help prevent thrombotic emboli or any parts thereof from migrating downstream due to antegrade flow during positioning of the intervention device 15 or filling of the aneurysm 10. The intervention device 15 is then advanced further into the vasculature so that a distal region is at or near the aneurysm 10, as shown in FIG. 9. During any part of the procedure, reverse flow may be initiated in the vessel via a retrograde flow system (described below) and/or via active aspiration.

FIGS. 9A and 9B show enlarged views of a distal region of the exemplary intervention device 15 positioned near the aneurysm 10. In FIG. 9A, the coil 58 is pushed by the pusher 60 from the catheter 52 into the aneurysm sac 70 through aneurysm neck 72. The detachable coil 58 forms a secondary loop after it leaves the end of the catheter 52. The most distal end 74 of the detachable coil 58 may also have an end plug or tip of some type simply to prevent punctures of the aneurysm as it is introduced into the aneurysm sac. The detachable coil 58 may be prebiased to form a cylinder or a conical envelope. The coil may be heat treated or crimped or otherwise physically treated to form a random shape after it is ejected from the catheter.

It is desirable that a significant volume of the aneurysm be filled with the coil 58. Consequently, the coil 58 may be quite flexible so to allow its conformance to the inner wall of the aneurysm 10 without puncture. In any event, once the coil 58 is properly placed within the aneurysm 10 and the attending physician positions the electrode 68 so to trim a proper amount of the detachable coil 58 into the aneurysm, a modest voltage is then applied to the device. In particular, a positive electric current of approximately 0.1 to 2 milliamps at 0.1 to 5.0 volts is applied to core wire 60 so to form a thrombus within the aneurysm sac 70. The negative pole of power supply 64 is attached to the conductor passing through or along the pusher 60.

The presence of the coil 58 causes a thrombus 76 to form and occlude the aneurysm 10, as shown in FIG. 9B. After the thrombus 76 has been formed and the aneurysm occluded, the core wire 60 with its electrode 68 is withdrawn as is the distal end of the catheter. This removal typically takes place within three to ten minutes, leaving aneurysm sac 130 occluded as is shown in FIG. 9B.

In another embodiment, the procedure involves the introduction into the aneurysm of a solid endovascular implant such as a coil or braid and a polymeric composition which may be reformed or solidified in situ for stabilizing and at least partially filling the aneurysm. The solid endovascular implant is at least partially surrounded or enveloped by the polymeric composition. The polymeric composition is reformed via light, heat, R.F. or the like to form a rigid mass with the solid endovascular implant. These steps may be carried out sequentially or the steps of introducing the endovascular implant and reforming the polymeric composition may be carried out simultaneously. The procedure may be accomplished using an intravascular catheter similar to the catheter 52 to access the desired site and to deliver the noted materials.

In another embodiment, the intervention device 15 is an embolic system which can deliver an embolic material or fluid composition through a microcatheter into the blood vessel. The material or composition solidifies and/or expands to fully or partially occlude the vascular site. The term “embolizing” or “embolization” refers to a process wherein a material or fluid composition is injected into a blood vessel which, in the case of, for example, aneurysms, fills or plugs the aneurysm sac and/or encourages clot formation so that blood flow into the aneurysm and pressure in the aneurysm ceases, and in the case of arterial venous malformations (AVMs) and arterial venous fistula (AVFs) forms a plug or clot to control/reroute blood flow to permit proper tissue perfusion. Embolization may be used for preventing/controlling bleeding due to lesions (e.g., organ bleeding, gastrointestinal bleeding, vascular bleeding, as well as bleeding associated with an aneurysm). In addition, embolization can be used to ablate diseased tissue (e.g., tumors, etc.) by cutting off its blood supply. U.S. Pat. Nos. 6,146,373 and 5,443,454 (which are both are incorporated herein by reference) describe exemplary liquid embolic systems.

In another embodiment, the intervention device 15 is a microcatheter used to delivery therapeutic agents such as cerebral protective agents, chemotherapeutic agents, stem cell or other regenerative agents, neurochemical or neuropsychopharmacologic agents, or the like, to an intracranial or cerebral artery and/or the brain.

In another embodiment, the intervention device 15 is a stent delivery catheter that is adapted to deliver a stent to a treatment location. In yet another embodiment, the intervention device 15 is a balloon dilatation or balloon occlusion catheter. In yet another embodiment, the intervention device 15 is a thrombus removal system. In yet another embodiment, the intervention device 15 is a brain tumor treatment device.

### Exemplary Embodiment of Retrograde Blood Flow System

As discussed, the system 100 includes the arterial access device 110, venous return device 115, and shunt 120 which provides a passageway for retrograde flow from the arterial access device 110 to the venous return device 115. The system also includes the flow control assembly 125, which interacts with the shunt 120 to regulate and/or monitor retrograde blood flow through the shunt 120. Exemplary embodiments of the components of the system 100 are now described.

Arterial Access Device

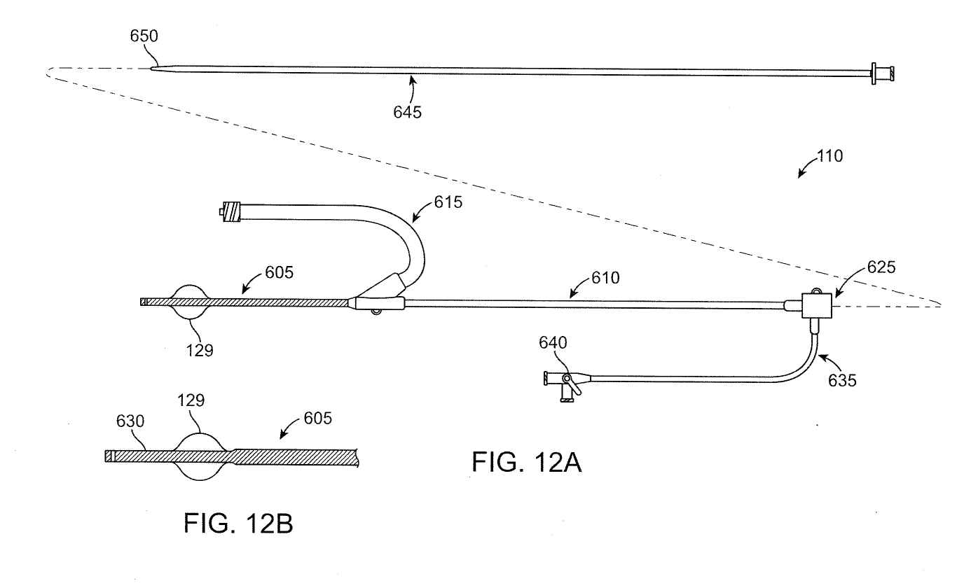

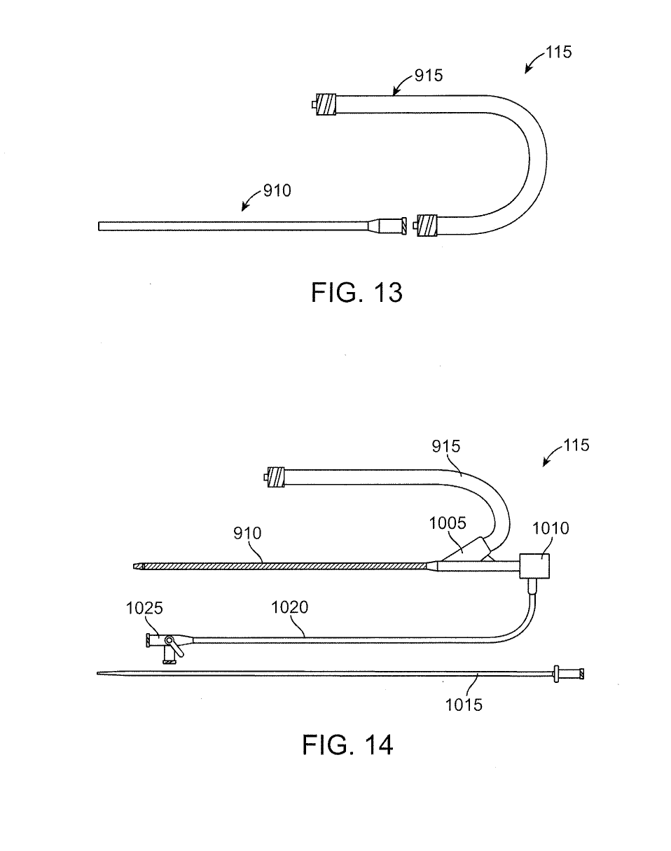

FIG. 10A shows an exemplary embodiment of the arterial access device 110, which comprises a distal sheath 605, a proximal extension 610, a flow line 615, an adaptor or Y-connector 620, and a hemostasis valve 625. The distal sheath 605 is adapted to be introduced through an incision or puncture in a wall of an artery such as a common carotid artery or vertebral artery, either an open surgical incision or a percutaneous puncture established, for example, using the Seldinger technique. The length of the sheath can be in the range from 5 to 15 cm, usually being from 10 cm to 12 cm. The inner diameter is typically in the range from 7 Fr (1 Fr=0.33 mm), to 10 Fr, usually being 8 Fr. Particularly when the sheath is being introduced through the transcervical approach, above the clavicle but below the carotid bifurcation, it is desirable that the sheath 605 be highly flexible while retaining hoop strength to resist kinking and buckling. Thus, the distal sheath 605 can be circumferentially reinforced, such as by braid, helical ribbon, helical wire, or the like. In an alternate embodiment, the distal sheath is adapted to be introduced through a percutaneous puncture into the femoral artery, such as in the groin, and up the aortic arch into the target artery.

The distal sheath 605 can have a stepped or other configuration having a reduced diameter distal region 630, as shown in FIG. 10B, which shows an enlarged view of the distal region 630 of the sheath 605. The distal region 630 of the sheath can be sized for insertion into the carotid artery, typically having an inner diameter in the range from 2.16 mm (0.085 inch) to 2.92 mm (0.115 inch) with the remaining proximal region of the sheath having larger outside and luminal diameters, with the inner diameter typically being in the range from 2.794 mm (0.110 inch) to 3.43 mm (0.135 inch). The larger luminal diameter of the proximal region minimizes the overall flow resistance of the sheath. In an embodiment, the reduced-diameter distal section 630 has a length of approximately 2 cm to 4 cm or 3 to 5 cm. In another embodiment, the reduced-diameter distal section 630 has a length of approximately 10 cm to 15 cm. The relatively short length of the reduced-diameter distal section 630 permits this section to be positioned in the common carotid artery CCA via the transcervical approach with reduced risk that the distal end of the sheath 605 will contact the bifurcation B. Moreover, the reduced diameter section 630 also permits a reduction in size of the arteriotomy for introducing the sheath 605 into the artery while having a minimal impact in the level of flow resistance.

With reference again to FIG. 10A, the proximal extension 610 has an inner lumen which is contiguous with an inner lumen of the sheath 605. The lumens can be joined by the Y-connector 620 which also connects a lumen of the flow line 615 to the sheath. In the assembled system, the flow line 615 connects to and forms a first leg of the retrograde shunt 120 (FIG. 5A). The proximal extension 610 can have a length sufficient to space the hemostasis valve 625 well away from the Y-connector 620, which is adjacent to the percutaneous or surgical insertion site. By spacing the hemostasis valve 625 away from a percutaneous insertion site, the physician can introduce a stent delivery system or other working catheter into the proximal extension 610 and sheath 605 while staying out of the fluoroscopic field when fluoroscopy is being performed.

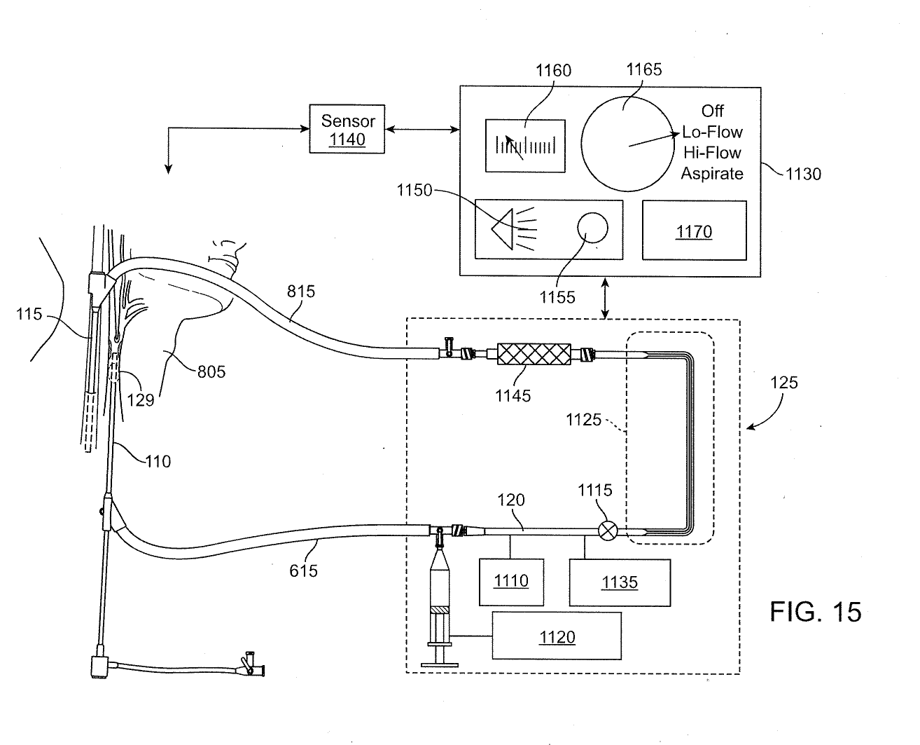

A flush line 635 can be connected to the side of the hemostasis valve 625 and can have a stopcock 640 at its proximal or remote end. The flush-line 635 allows for the introduction of saline, contrast fluid, or the like, during the procedures. The flush line 635 can also allow pressure monitoring during the procedure. A dilator 645 having a tapered distal end 650 can be provided to facilitate introduction of the distal sheath 605 into the common carotid artery. The dilator 645 can be introduced through the hemostasis valve 625 so that the tapered distal end 650 extends through the distal end of the sheath 605, as best seen in FIG. 11A. The dilator 645 can have a central lumen to accommodate a guide wire. Typically, the guide wire is placed first into the vessel, and the dilator/sheath combination travels over the guide wire as it is being introduced into the vessel.

Optionally, a tube 705 may be provided which is coaxially received over the exterior of the distal sheath 605, also as seen in FIG. 11A. The tube 705 has a flared proximal end 710 which engages the adapter 620 and a distal end 715. Optionally, the distal end 715 may be beveled, as shown in FIG. 11B. The tube 705 may serve at least two purposes. First, the length of the tube 705 limits the introduction of the sheath 605 to the exposed distal portion of the sheath 605, as seen in FIG. 11A. Second, the tube 705 can engage a pre-deployed puncture closure device disposed in the carotid artery wall, if present, to permit the sheath 605 to be withdrawn without dislodging the closure device.

The distal sheath 605 can be configured to establish a curved transition from a generally anterior-posterior approach over the common carotid artery to a generally axial luminal direction within the common carotid artery. The transition in direction is particularly useful when a percutaneous access is provided through the common carotid wall. While an open surgical access may allow for some distance in which to angle a straight sheath into the lumen of the common carotid artery, percutaneous access will generally be in a normal or perpendicular direction relative to the access of the lumen, and in such cases, a sheath that can flex or turn at an angle will find great use.

The sheath 605 can be formed in a variety of ways. For example, the sheath 605 can be pre-shaped to have a curve or an angle some set distance from the tip, typically 2 to 3 cm. The pre-shaped curve or angle can typically provide for a turn in the range from 20° to 90°, preferably from 30° to 70°. For initial introduction, the sheath 605 can be straightened with an obturator or other straight or shaped instrument such as the dilator 645 placed into its lumen. After the sheath 605 has been at least partially introduced through the percutaneous or other arterial wall penetration, the obturator can be withdrawn to allow the sheath 605 to reassume its pre-shaped configuration into the arterial lumen.

Other sheath configurations include having a deflection mechanism such that the sheath can be placed and the catheter can be deflected in situ to the desired deployment angle. In still other configurations, the catheter has a non-rigid configuration when placed into the lumen of the common carotid artery. Once in place, a pull wire or other stiffening mechanism can be deployed in order to shape and stiffen the sheath into its desired configuration. One particular example of such a mechanism is commonly known as “shape-lock” mechanisms as well described in medical and patent literature.

Another sheath configuration comprises a curved dilator inserted into a straight but flexible sheath, so that the dilator and sheath are curved during insertion. The sheath is flexible enough to conform to the anatomy after dilator removal.

In an embodiment, the sheath has built-in puncturing capability and atraumatic tip analogous to a guide wire tip. This eliminates the need for needle and wire exchange currently used for arterial access according to the micropuncture technique, and can thus save time, reduce blood loss, and require less surgeon skill.

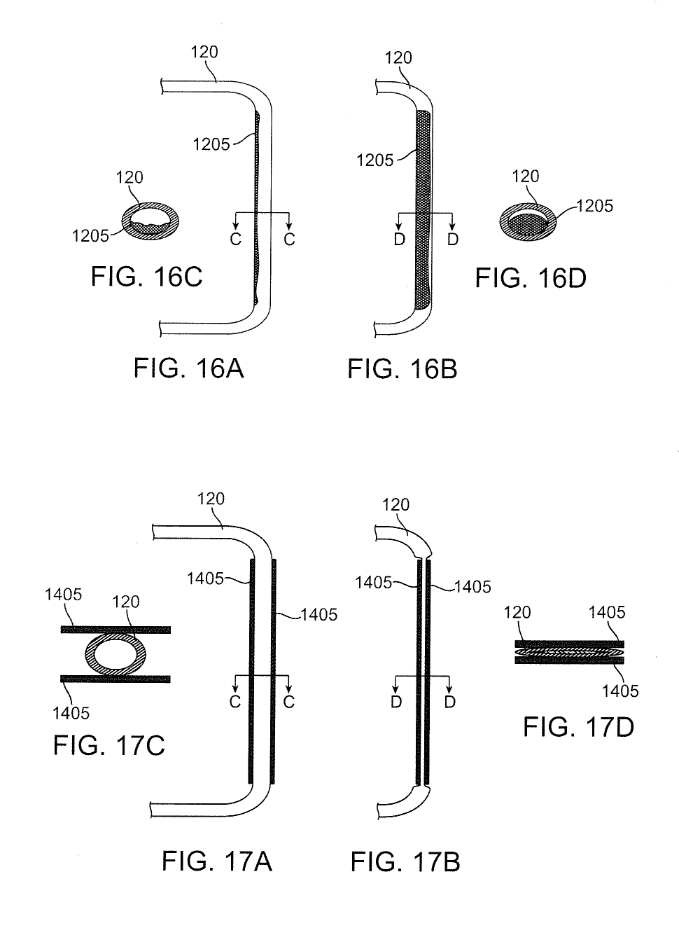

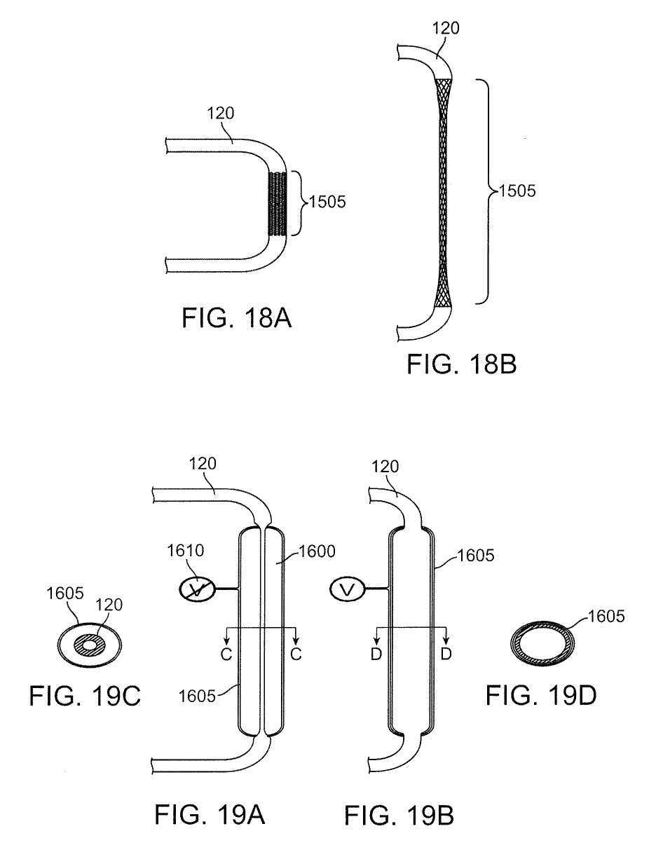

FIG. 12A shows another embodiment of the arterial access device 110. This embodiment is substantially the same as the embodiment shown in FIG. 10A, except that the distal sheath 605 includes an occlusion element 129 for occluding flow through, for example the common carotid artery. If the occluding element 129 is an inflatable structure such as a balloon or the like, the sheath 605 can include an inflation lumen that communicates with the occlusion element 129. The occlusion element 129 can be an inflatable balloon, but it could also be an inflatable cuff, a conical or other circumferential element which flares outwardly to engage the interior wall of the common or internal carotid artery to block flow therepast, a membrane-covered braid, a slotted tube that radially enlarges when axially compressed, or similar structure which can be deployed by mechanical means, or the like. In the case of balloon occlusion, the balloon can be compliant, non-compliant, elastomeric, reinforced, or have a variety of other characteristics. In an embodiment, the balloon is an elastomeric balloon which is closely received over the exterior of the distal end of the sheath prior to inflation. When inflated, the elastomeric balloon can expand and conform to the inner wall of the common carotid artery. In an embodiment, the elastomeric balloon is able to expand to a diameter at least twice that of the non-deployed configuration, frequently being able to be deployed to a diameter at least three times that of the undeployed configuration, more preferably being at least four times that of the undeployed configuration, or larger.

As shown in FIG. 12B, the distal sheath 605 with the occlusion element 129 can have a stepped or other configuration having a reduced diameter distal region 630. The distal region 630 can be sized for insertion into the carotid artery with the remaining proximal region of the sheath 605 having larger outside and luminal diameters, with the inner diameter typically being in the range from 2.794 mm (0.110 inch) to 3.43 mm (0.135 inch). The larger luminal diameter of the proximal region minimizes the overall flow resistance of the sheath. In an embodiment, the reduced-diameter distal section 630 has a length of approximately 2 cm to 4 cm. The relatively short length of the reduced-diameter distal section 630 permits this section to be positioned in the common carotid artery CCA via the transcervical approach with reduced risk that the distal end of the sheath 605 will contact the bifurcation B.

Venous Return Device

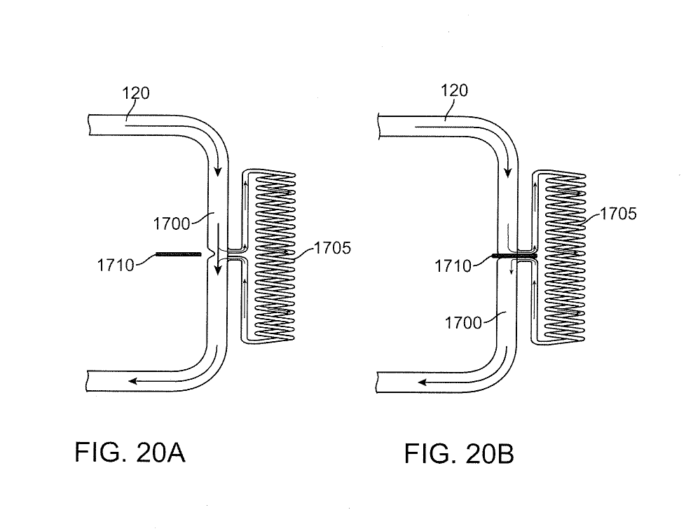

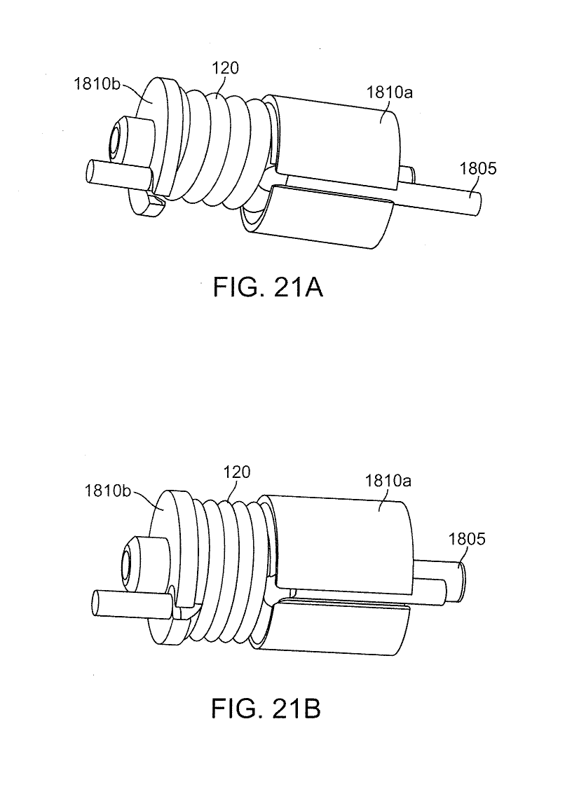

Referring now to FIG. 13, the venous return device 115 can comprise a distal sheath 910 and a flow line 915, which connects to and forms a leg of the shunt 120 when the system is in use. The distal sheath 910 is adapted to be introduced through an incision or puncture into a venous return location, such as the jugular vein or femoral vein. The distal sheath 910 and flow line 915 can be permanently affixed, or can be attached using a conventional luer fitting, as shown in FIG. 13. Optionally, as shown in FIG. 14, the sheath 910 can be joined to the flow line 915 by a Y-connector 1005. The Y-connector 1005 can include a hemostasis valve 1010, permitting insertion of a dilator 1015 to facilitate introduction of the venous return device into the internal jugular vein or other vein. As with the arterial access dilator 645, the venous dilator 1015 includes a central guide wire lumen so the venous sheath and dilator combination can be placed over a guide wire. Optionally, the venous sheath 910 can include a flush line 1020 with a stopcock 1025 at its proximal or remote end.

In order to reduce the overall system flow resistance, the arterial access flow line 615 and Y-connector 620 (FIG. 10A) and the venous return flow line 915, and Y-connectors 1005 (FIG. 13 or 14), can each have a relatively large flow lumen inner diameter, typically being in the range from 2.54 mm (0.100 inch) to 5.08 mm (0.200 inch), and a relatively short length, typically being in the range from 10 cm to 20 cm. The low system flow resistance is desirable since it permits the flow to be maximized during portions of a procedure when the risk of emboli is at its greatest. The low system flow resistance also allows the use of a variable flow resistance for controlling flow in the system, as described in more detail below. The dimensions of the venous return sheath 910 can be generally the same as those described for the arterial access sheath 605 above. In the venous return sheath, an extension for the hemostasis valve 1010 is not required.

Retrograde Shunt

The shunt 120 can be formed of a single tube or multiple, connected tubes that provide fluid communication between the arterial access catheter 110 and the venous return catheter 115 to provide a pathway for retrograde blood flow therebetween. As shown in FIG. 5A, the shunt 120 connects at one end to the flow line 615 of the arterial access device 110, and at an opposite end to the flow line 915 of the venous return catheter 115.

In an embodiment, the shunt 120 can be formed of at least one tube that communicates with the flow control assembly 125. The shunt 120 can be any structure that provides a fluid pathway for blood flow. The shunt 120 can have a single lumen or it can have multiple lumens. The shunt 120 can be removably attached to the flow control assembly 125, arterial access device 110, and/or venous return device 115. Prior to use, the user can select a shunt 120 with a length that is most appropriate for use with the arterial access location and venous return location. In an embodiment, the shunt 120 can include one or more extension tubes that can be used to vary the length of the shunt 120. The extension tubes can be modularly attached to the shunt 120 to achieve a desired length. The modular aspect of the shunt 120 permits the user to lengthen the shunt 120 as needed depending on the site of venous return. For example, in some patients, the internal jugular vein IJV is small and/or tortuous. The risk of complications at this site may be higher than at some other locations, due to proximity to other anatomic structures. In addition, hematoma in the neck may lead to airway obstruction and/or cerebral vascular complications. Consequently, for such patients it may be desirable to locate the venous return site at a location other than the internal jugular vein IJV, such as the femoral vein. A femoral vein return site may be accomplished percutaneously, with lower risk of serious complication, and also offers an alternative venous access to the central vein if the internal jugular vein IJV is not available. Furthermore, the femoral venous return changes the layout of the reverse flow shunt such that the shunt controls may be located closer to the “working area” of the intervention, where the devices are being introduced and the contrast injection port is located.

In an embodiment, the shunt 120 has an internal diameter of 4.76 mm ( 3/16 inch) and has a length of 40-70 cm. As mentioned, the length of the shunt can be adjusted.

In an embodiment, the shunt may contain a port which can be connected to an aspiration source such as a syringe, suction pump, or the like.

In an additional embodiment, the shunt may contain an element that connects to an active pump, for example a peristaltic pump, a diaphragm pump, an impeller pump, or a syringe pump.

Flow Control Assembly—Regulation and Monitoring of Retrograde Flow

The flow control assembly 125 interacts with the retrograde shunt 120 to regulate and/or monitor the retrograde flow rate from the common carotid artery to the venous return site, such as the internal jugular vein, or to the external receptacle. In this regard, the flow control assembly 125 enables the user to achieve higher maximum flow rates than existing systems and to also selectively adjust, set, or otherwise modulate the retrograde flow rate. Various mechanisms can be used to regulate the retrograde flow rate, as described more fully below. The flow control assembly 125 enables the user to configure retrograde blood flow in a manner that is suited for various treatment regimens, as described below.

In general, the ability to control the continuous retrograde flow rate allows the physician to adjust the protocol for individual patients and stages of the procedure. The retrograde blood flow rate will typically be controlled over a range from a low rate to a high rate. The high rate can be at least two fold higher than the low rate, typically being at least three fold higher than the low rate, and often being at least five fold higher than the low rate, or even higher. In an embodiment, the high rate is at least three fold higher than the low rate and in another embodiment the high rate is at least six fold higher than the low rate. While it is generally desirable to have a high retrograde blood flow rate to maximize the extraction of emboli from the carotid arteries, the ability of patients to tolerate retrograde blood flow will vary. Thus, by having a system and protocol which allows the retrograde blood flow rate to be easily modulated, the treating physician can determine when the flow rate exceeds the tolerable level for that patient and set the reverse flow rate accordingly. For patients who cannot tolerate continuous high reverse flow rates, the physician can chose to turn on high flow only for brief, critical portions of the procedure when the risk of embolic debris is highest. At short intervals, for example between 15 seconds and 1 minute, patient tolerance limitations are usually not a factor.

In specific embodiments, the continuous retrograde blood flow rate can be controlled at a base line flow rate in the range from 10 ml/min to 200 ml/min, typically from 20 ml/min to 100 ml/min. These flow rates will be tolerable to the majority of patients. Although flow rate is maintained at the base line flow rate during most of the procedure, at times when the risk of emboli release is increased, the flow rate can be increased above the base line for a short duration in order to improve the ability to capture such emboli. For example, the retrograde blood flow rate can be increased above the base line when the stent catheter is being introduced, when the stent is being deployed, pre- and post-dilatation of the stem, removal of the common carotid artery occlusion, and the like.

The flow rate control system can be cycled between a relatively low flow rate and a relatively high flow rate in order to “flush” the carotid arteries in the region of the carotid bifurcation prior to reestablishing antegrade flow. Such cycling can be established with a high flow rate which can be approximately two to six fold greater than the low flow rate, typically being about three fold greater. The cycles can typically have a length in the range from 0.5 seconds to 10 seconds, usually from 2 seconds to 5 seconds, with the total duration of the cycling being in the range from 5 seconds to 60 seconds, usually from 10 seconds to 30 seconds.

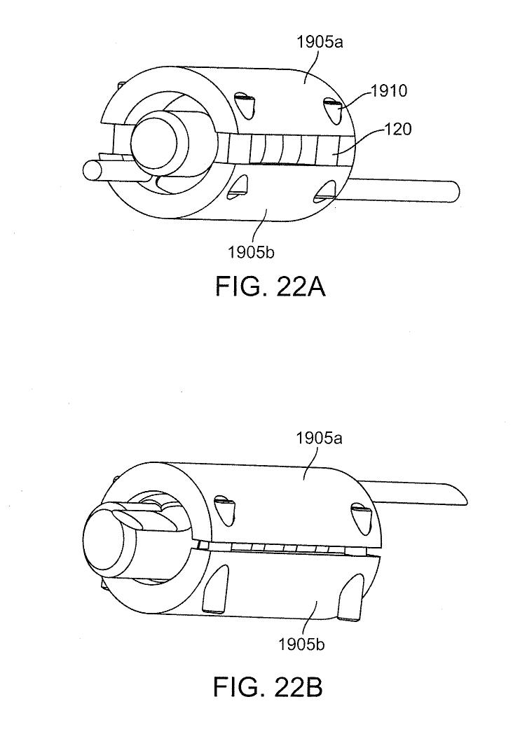

FIG. 15 shows an example of the system 100 with a schematic representation of the flow control assembly 125, which is positioned along the shunt 120 such that retrograde blood flow passes through or otherwise communicates with at least a portion of the flow control assembly 125. The flow control assembly 125 can include various controllable mechanisms for regulating and/or monitoring retrograde flow. The mechanisms can include various means of controlling the retrograde flow, including one or more pumps 1110, valves 1115, syringes 1120 and/or a variable resistance component 1125. The flow control assembly 125 can be manually controlled by a user and/or automatically controlled via a controller 1130 to vary the flow through the shunt 120. For example, varying the flow resistance, the rate of retrograde blood flow through the shunt 120 can be controlled. The controller 1130, which is described in more detail below, can be integrated into the flow control assembly 125 or it can be a separate component that communicates with the components of the flow control assembly 125.

In addition, the flow control assembly 125 can include one or more flow sensors 1135 and/or anatomical data sensors 1140 (described in detail below) for sensing one or more aspects of the retrograde flow. A filter 1145 can be positioned along the shunt 120 for removing emboli before the blood is returned to the venous return site. When the filter 1145 is positioned upstream of the controller 1130, the filter 1145 can prevent emboli from entering the controller 1145 and potentially clogging the variable flow resistance component 1125. It should be appreciated that the various components of the flow control assembly 125 (including the pump 1110, valves 1115, syringes 1120, variable resistance component 1125, sensors 1135/1140, and filter 1145) can be positioned at various locations along the shunt 120 and at various upstream or downstream locations relative to one another. The components of the flow control assembly 125 are not limited to the locations shown in FIG. 15. Moreover, the flow control assembly 125 does not necessarily include all of the components but can rather include various sub-combinations of the components. For example, a syringe could optionally be used within the flow control assembly 125 for purposes of regulating flow or it could be used outside of the assembly for purposes other than flow regulation, such as to introduce fluid such as radiopaque contrast into the artery in an antegrade direction via the shunt 120.

Both the variable resistance component 1125 and the pump 1110 can be coupled to the shunt 120 to control the retrograde flow rate. The variable resistance component 1125 controls the flow resistance, while the pump 1110 provides for positive displacement of the blood through the shunt 120. Thus, the pump can be activated to drive the retrograde flow rather than relying on the perfusion stump pressures of the ECA and ICA and the venous back pressure to drive the retrograde flow. The pump 1110 can be a peristaltic tube pump or any type of pump including a positive displacement pump. The pump 1110 can be activated and deactivated (either manually or automatically via the controller 1130) to selectively achieve blood displacement through the shunt 120 and to control the flow rate through the shunt 120. Displacement of the blood through the shunt 120 can also be achieved in other manners including using the aspiration syringe 1120, or a suction source such as a vacutainer, vaculock syringe, or wall suction may be used. The pump 1110 can communicate with the controller 1130.

One or more flow control valves 1115 can be positioned along the pathway of the shunt. The valve(s) can be manually actuated or automatically actuated (via the controller 1130). The flow control valves 1115 can be, for example one-way valves to prevent flow in the antegrade direction in the shunt 120, check valves, or high pressure valves which would close off the shunt 120, for example during high-pressure contrast injections (which are intended to enter the arterial vasculature in an antegrade direction).

The controller 1130 communicates with components of the system 100 including the flow control assembly 125 to enable manual and/or automatic regulation and/or monitoring of the retrograde flow through the components of the system 100 (including, for example, the shunt 120, the arterial access device 110, the venous return device 115 and the flow control assembly 125). For example, a user can actuate one or more actuators on the controller 1130 to manually control the components of the flow control assembly 125. Manual controls can include switches or dials or similar components located directly on the controller 1130 or components located remote from the controller 1130 such as a foot pedal or similar device. The controller 1130 can also automatically control the components of the system 100 without requiring input from the user. In an embodiment, the user can program software in the controller 1130 to enable such automatic control. The controller 1130 can control actuation of the mechanical portions of the flow control assembly 125. The controller 1130 can include circuitry or programming that interprets signals generated by sensors 1135/1140 such that the controller 1130 can control actuation of the flow control assembly 125 in response to such signals generated by the sensors.

The flow control assembly 125 may also include an active pump actuator which interfaces with an element in the shunt to enable active retrograde pumping of blood, such as a pump head for a roller pump, a rotary motor for an impeller-style pump, or the like. The controller 1130 would provide controls for the pump rate.

The representation of the controller 1130 in FIG. 15 is merely exemplary. It should be appreciated that the controller 1130 can vary in appearance and structure. The controller 1130 is shown in FIG. 15 as being integrated in a single housing. This permits the user to control the flow control assembly 125 from a single location. It should be appreciated that any of the components of the controller 1130 can be separated into separate housings. Further, FIG. 15 shows the controller 1130 and flow control assembly 125 as separate housings. It should be appreciated that the controller 1130 and flow control regulator 125 can be integrated into a single housing or can be divided into multiple housings or components.

Flow State Indicator(s)

The controller 1130 can include one or more indicators that provides a visual and/or audio signal to the user regarding the state of the retrograde flow. An audio indication advantageously reminds the user of a flow state without requiring the user to visually check the flow controller 1130. The indicator(s) can include a speaker 1150 and/or a light 1155 or any other means for communicating the state of retrograde flow to the user. The controller 1130 can communicate with one or more sensors of the system to control activation of the indicator. Or, activation of the indicator can be tied directly to the user actuating one of the flow control actuators 1165. The indicator need not be a speaker or a light. The indicator could simply be a button or switch that visually indicates the state of the retrograde flow. For example, the button being in a certain state (such as a pressed or down state) may be a visual indication that the retrograde flow is in a high state. Or, a switch or dial pointing toward a particular labeled flow state may be a visual indication that the retrograde flow is in the labeled state.

The indicator can provide a signal indicative of one or more states of the retrograde flow. In an embodiment, the indicator identifies only two discrete states: a state of “high” flow rate and a state of “low” flow rate. In another embodiment, the indicator identifies more than two flow rates, including a “high” flow rate, a “medium” flow rate, and a “low” rate. The indicator can be configured to identify any quantity of discrete states of the retrograde flow or it can identify a graduated signal that corresponds to the state of the retrograde flow. In this regard, the indicator can be a digital or analog meter 1160 that indicates a value of the retrograde flow rate, such as in ml/min or any other units.

In an embodiment, the indicator is configured to indicate to the user whether the retrograde flow rate is in a state of “high” flow rate or a “low” flow rate. For example, the indicator may illuminate in a first manner (e.g., level of brightness) and/or emit a first audio signal when the flow rate is high and then change to a second manner of illumination and/or emit a second audio signal when the flow rate is low. Or, the indicator may illuminate and/or emit an audio signal only when the flow rate is high, or only when the flow rate is low. Given that some patients may be intolerant of a high flow rate or intolerant of a high flow rate beyond an extended period of time, it can be desirable that the indicator provide notification to the user when the flow rate is in the high state. This would serve as a fail safe feature.

In another embodiment, the indicator provides a signal (audio and/or visual) when the flow rate changes state, such as when the flow rate changes from high to low and/or vice-versa. In another embodiment, the indicator provides a signal when no retrograde flow is present, such as when the shunt 120 is blocked or one of the stopcocks in the shunt 120 is closed.

Flow Rate Actuators

The controller 1130 can include one or more actuators that the user can press, switch, manipulate, or otherwise actuate to regulate the retrograde flow rate and/or to monitor the flow rate. For example, the controller 1130 can include a flow control actuator 1165 (such as one or more buttons, knobs, dials, switches, etc.) that the user can actuate to cause the controller to selectively vary an aspect of the reverse flow. For example, in the illustrated embodiment, the flow control actuator 1165 is a knob that can be turned to various discrete positions each of which corresponds to the controller 1130 causing the system 100 to achieve a particular retrograde flow state. The states include, for example, (a) OFF; (b) LO-FLOW; (c) HI-FLOW; and (d) ASPIRATE. It should be appreciated that the foregoing states are merely exemplary and that different states or combinations of states can be used. The controller 1130 achieves the various retrograde flow states by interacting with one or more components of the system, including the sensor(s), valve(s), variable resistance component, and/or pump(s). It should be appreciated that the controller 1130 can also include circuitry and software that regulates the retrograde flow rate and/or monitors the flow rate such that the user wouldn't need to actively actuate the controller 1130.

The OFF state corresponds to a state where there is no retrograde blood flow through the shunt 120. When the user sets the flow control actuator 1165 to OFF, the controller 1130 causes the retrograde flow to cease, such as by shutting off valves or closing a stop cock in the shunt 120. The LO-FLOW and HI-FLOW states correspond to a low retrograde flow rate and a high retrograde flow rate, respectively. When the user sets the flow control actuator 1165 to LO-FLOW or HI-FLOW, the controller 1130 interacts with components of the flow control regulator 125 including pump(s) 1110, valve(s) 1115 and/or variable resistance component 1125 to increase or decrease the flow rate accordingly. Finally, the ASPIRATE state corresponds to opening the circuit to a suction source, for example a vacutainer or suction unit, if active retrograde flow is desired. The suction source can be coupled to any portion of the circuit, including the shunt 120 or the arterial access device 110.

The system can be used to vary the blood flow between various states including an active state, a passive state, an aspiration state, and an off state. The active state corresponds to the system using a means that actively drives retrograde blood flow. Such active means can include, for example, a pump, syringe, vacuum source, etc. The passive state corresponds to when retrograde blood flow is driven by the perfusion stump pressures of the ECA and ICA and possibly the venous pressure. The aspiration state corresponds to the system using a suction source, for example a vacutainer or suction unit, to drive retrograde blood flow. The off state corresponds to the system having zero retrograde blood flow such as the result of closing a stopcock or valve. The low and high flow rates can be either passive or active flow states. In an embodiment, the particular value (such as in ml/min) of either the low flow rate and/or the high flow rate can be predetermined and/or pre-programmed into the controller such that the user does not actually set or input the value. Rather, the user simply selects “high flow” and/or “low flow” (such as by pressing an actuator such as a button on the controller 1130) and the controller 1130 interacts with one or more of the components of the flow control assembly 125 to cause the flow rate to achieve the predetermined high or low flow rate value. In another embodiment, the user sets or inputs a value for low flow rate and/or high flow rate such as into the controller. In another embodiment, the low flow rate and/or high flow rate is not actually set. Rather, external data (such as data from the anatomical data sensor 1140) is used as the basis for affects the flow rate.

The flow control actuator 1165 can be multiple actuators, for example one actuator, such as a button or switch, to switch state from LO-FLOW to HI-FLOW and another to close the flow loop to OFF, for example during a contrast injection where the contrast is directed antegrade into the carotid artery. In an embodiment, the flow control actuator 1165 can include multiple actuators. For example, one actuator can be operated to switch flow rate from low to high, another actuator can be operated to temporarily stop flow, and a third actuator (such as a stopcock) can be operated for aspiration using a syringe. In another example, one actuator is operated to switch to LO-FLOW and another actuator is operated to switch to HI-FLOW. Or, the flow control actuator 1165 can include multiple actuators to switch states from LO-FLOW to HI-FLOW and additional actuators for fine-tuning flow rate within the high flow state and low flow state. Upon switching between LO-FLOW and HI-FLOW, these additional actuators can be used to fine-tune the flow rates within those states. Thus, it should be appreciated that within each state (i.e. high flow state and low flow states) a variety of flow rates can be dialed in and fine-tuned. A wide variety of actuators can be used to achieve control over the state of flow.

The controller 1130 or individual components of the controller 1130 can be located at various positions relative to the patient and/or relative to the other components of the system 100. For example, the flow control actuator 1165 can be located near the hemostasis valve where any interventional tools are introduced into the patient in order to facilitate access to the flow control actuator 1165 during introduction of the tools. The location may vary, for example, based on whether a transfemoral or a transcervical approach is used. The controller 1130 can have a wireless connection to the remainder of the system 100 and/or a wired connection of adjustable length to permit remote control of the system 100. The controller 1130 can have a wireless connection with the flow control regulator 125 and/or a wired connection of adjustable length to permit remote control of the flow control regulator 125. The controller 1130 can also be integrated in the flow control regulator 125. Where the controller 1130 is mechanically connected to the components of the flow control assembly 125, a tether with mechanical actuation capabilities can connect the controller 1130 to one or more of the components. In an embodiment, the controller 1130 can be positioned a sufficient distance from the system 100 to permit positioning the controller 1130 outside of a radiation field when fluoroscopy is in use.

The controller 1130 and any of its components can interact with other components of the system (such as the pump(s), sensor(s), shunt, etc) in various manners. For example, any of a variety of mechanical connections can be used to enable communication between the controller 1130 and the system components. Alternately, the controller 1130 can communicate electronically or magnetically with the system components. Electro-mechanical connections can also be used. The controller 1130 can be equipped with control software that enables the controller to implement control functions with the system components. The controller itself can be a mechanical, electrical or electro-mechanical device. The controller can be mechanically, pneumatically, or hydraulically actuated or electromechanically actuated (for example in the case of solenoid actuation of flow control state). The controller 1130 can include a computer, computer processor, and memory, as well as data storage capabilities.

Sensor(s)

As mentioned, the flow control assembly 125 can include or interact with one or more sensors, which communicate with the system 100 and/or communicate with the patient's anatomy. Each of the sensors can be adapted to respond to a physical stimulus (including, for example, heat, light, sound, pressure, magnetism, motion, etc.) and to transmit a resulting signal for measurement or display or for operating the controller 1130. In an embodiment, the flow sensor 1135 interacts with the shunt 120 to sense an aspect of the flow through the shunt 120, such as flow velocity or volumetric rate of blood flow. The flow sensor 1135 could be directly coupled to a display that directly displays the value of the volumetric flow rate or the flow velocity. Or the flow sensor 1135 could feed data to the controller 1130 for display of the volumetric flow rate or the flow velocity.

The type of flow sensor 1135 can vary. The flow sensor 1135 can be a mechanical device, such as a paddle wheel, flapper valve, rolling ball, or any mechanical component that responds to the flow through the shunt 120. Movement of the mechanical device in response to flow through the shunt 120 can serve as a visual indication of fluid flow and can also be calibrated to a scale as a visual indication of fluid flow rate. The mechanical device can be coupled to an electrical component. For example, a paddle wheel can be positioned in the shunt 120 such that fluid flow causes the paddle wheel to rotate, with greater rate of fluid flow causing a greater speed of rotation of the paddle wheel. The paddle wheel can be coupled magnetically to a Hall-effect sensor to detect the speed of rotation, which is indicative of the fluid flow rate through the shunt 120.

In an embodiment, the flow sensor 1135 is an ultrasonic or electromagnetic flow meter, which allows for blood flow measurement without contacting the blood through the wall of the shunt 120. An ultrasonic or electromagnetic flow meter can be configured such that it does not have to contact the internal lumen of the shunt 120. In an embodiment, the flow sensor 1135 at least partially includes a Doppler flow meter, such as a Transonic flow meter, that measures fluid flow through the shunt 120. It should be appreciated that any of a wide variety of sensor types can be used including an ultrasound flow meter and transducer. Moreover, the system can include multiple sensors.

The system 100 is not limited to using a flow sensor 1135 that is positioned in the shunt 120 or a sensor that interacts with the venous return device 115 or the arterial access device 110. For example, an anatomical data sensor 1140 can communicate with or otherwise interact with the patient's anatomy such as the patient's neurological anatomy. In this manner, the anatomical data sensor 1140 can sense a measurable anatomical aspect that is directly or indirectly related to the rate of retrograde flow from the carotid artery. For example, the anatomical data sensor 1140 can measure blood flow conditions in the brain, for example the flow velocity in the middle cerebral artery, and communicate such conditions to a display and/or to the controller 1130 for adjustment of the retrograde flow rate based on predetermined criteria. In an embodiment, the anatomical data sensor 1140 comprises a transcranial Doppler ultrasonography (TCD), which is an ultrasound test that uses reflected sound waves to evaluate blood as it flows through the brain. Use of TCD results in a TCD signal that can be communicated to the controller 1130 for controlling the retrograde flow rate to achieve or maintain a desired TCD profile. The anatomical data sensor 1140 can be based on any physiological measurement, including reverse flow rate, blood flow through the middle cerebral artery, TCD signals of embolic particles, or other neuromonitoring signals.

In an embodiment, the system 100 comprises a closed-loop control system. In the closed-loop control system, one or more of the sensors (such as the flow sensor 1135 or the anatomical data sensor 1140) senses or monitors a predetermined aspect of the system 100 or the anatomy (such as, for example, reverse flow rate and/or neuromonitoring signal). The sensor(s) feed relevant data to the controller 1130, which continuously adjusts an aspect of the system as necessary to maintain a desired retrograde flow rate. The sensors communicate feedback on how the system 100 is operating to the controller 1130 so that the controller 1130 can translate that data and actuate the components of the flow control regulator 125 to dynamically compensate for disturbances to the retrograde flow rate. For example, the controller 1130 may include software that causes the controller 1130 to signal the components of the flow control assembly 125 to adjust the flow rate such that the flow rate is maintained at a constant state despite differing blood pressures from the patient. In this embodiment, the system 100 need not rely on the user to determine when, how long, and/or what value to set the reverse flow rate in either a high or low state. Rather, software in the controller 1130 can govern such factors. In the closed loop system, the controller 1130 can control the components of the flow control assembly 125 to establish the level or state of retrograde flow (either analog level or discreet state such as high, low, baseline, medium, etc.) based on the retrograde flow rate sensed by the sensor 1135.

In an embodiment, the anatomical data sensor 1140 (which measures a physiologic measurement in the patient) communicates a signal to the controller 1130, which adjusts the flow rate based on the signal. For example the physiological measurement may be based on flow velocity through the MCA, TCD signal, or some other cerebral vascular signal. In the case of the TCD signal, TCD may be used to monitor cerebral flow changes and to detect microemboli. The controller 1130 may adjust the flow rate to maintain the TCD signal within a desired profile. For example, the TCD signal may indicate the presence of microemboli (“TCD hits”) and the controller 1130 can adjust the retrograde flow rate to maintain the TCD hits below a threshold value of hits. (See, Ribo, et al., “Transcranial Doppler Monitoring of Transcervical Carotid Stenting with Flow Reversal Protection: A Novel Carotid Revascularization Technique”, Stroke 2006, 37, 2846-2849; Shekel, et al., “Experience of 500 Cases of Neurophysiological Monitoring in Carotid Endarterectomy”, Acta Neurochir, 2007, 149:681-689, which are incorporated by reference in their entirety.)

In the case of the MCA flow, the controller 1130 can set the retrograde flow rate at the “maximum” flow rate that is tolerated by the patient, as assessed by perfusion to the brain. The controller 1130 can thus control the reverse flow rate to optimize the level of protection for the patient without relying on the user to intercede. In another embodiment, the feedback is based on a state of the devices in the system 100 or the interventional tools being used. For example, a sensor may notify the controller 1130 when the system 100 is in a high risk state, such as when an interventional catheter is positioned in the sheath 605. The controller 1130 then adjusts the flow rate to compensate for such a state.

The controller 1130 can be used to selectively augment the retrograde flow in a variety of manners. For example, it has been observed that greater reverse flow rates may cause a resultant greater drop in blood flow to the brain, most importantly the ipsilateral MCA, which may not be compensated enough with collateral flow from the Circle of Willis. Thus a higher reverse flow rate for an extended period of time may lead to conditions where the patient's brain is not getting enough blood flow, leading to patient intolerance as exhibited by neurologic symptoms. Studies show that MCA blood velocity less than 10 cm/sec is a threshold value below which patient is at risk for neurological blood deficit. There are other markers for monitoring adequate perfusion to the brains, such as EEG signals. However, a high flow rate may be tolerated even up to a complete stoppage of MCA flow for a short period, up to about 15 seconds to 1 minute.

Thus, the controller 1130 can optimize embolic debris capture by automatically increasing the reverse flow only during limited time periods which correspond to periods of heightened risk of emboli generation during a procedure. These periods of heightened risk include the period of time while an interventional device (such as the intervention device 15) crosses the aneurysm 10. During lower risk periods, the controller can cause the reverse flow rate to revert to a lower, baseline level. This lower level may correspond to a low reverse flow rate in the ICA, or even slight antegrade flow in those patients with a high ECA to ICA perfusion pressure ratio.

In a flow regulation system where the user manually sets the state of flow, there is risk that the user may not pay attention to the state of retrograde flow (high or low) and accidentally keep the circuit on high flow. This may then lead to adverse patient reactions. In an embodiment, as a safety mechanism, the default flow rate is the low flow rate. This serves as a fail safe measure for patient's that are intolerant of a high flow rate. In this regard, the controller 1130 can be biased toward the default rate such that the controller causes the system to revert to the low flow rate after passage of a predetermined period of time of high flow rate. The bias toward low flow rate can be achieved via electronics or software, or it can be achieved using mechanical components, or a combination thereof. In an embodiment, the flow control actuator 1165 of the controller 1130 and/or valve(s) 1115 and/or pump(s) 1110 of the flow control regulator 125 are spring loaded toward a state that achieves a low flow rate. The controller 1130 is configured such that the user may over-ride the controller 1130 such as to manually cause the system to revert to a state of low flow rate if desired.

In another safety mechanism, the controller 1130 includes a timer 1170 (FIG. 15) that keeps time with respect to how long the flow rate has been at a high flow rate. The controller 1130 can be programmed to automatically cause the system 100 to revert to a low flow rate after a predetermined time period of high flow rate, for example after 15, 30, or 60 seconds or more of high flow rate. After the controller reverts to the low flow rate, the user can initiate another predetermined period of high flow rate as desired. Moreover, the user can override the controller 1130 to cause the system 100 to move to the low flow rate (or high flow rate) as desired.

In an exemplary procedure, embolic debris capture is optimized while not causing patient tolerance issues by initially setting the level of retrograde flow at a low rate, and then switching to a high rate for discreet periods of time during critical stages in the procedure. Alternately, the flow rate is initially set at a high rate, and then verifying patient tolerance to that level before proceeding with the rest of the procedure. If the patient shows signs of intolerance, the retrograde flow rate is lowered. Patient tolerance may be determined automatically by the controller based on feedback from the anatomical data sensor 1140 or it may be determined by a user based on patient observation. The adjustments to the retrograde flow rate may be performed automatically by the controller or manually by the user. Alternately, the user may monitor the flow velocity through the middle cerebral artery (MCA), for example using TCD, and then to set the maximum level of reverse flow which keeps the MCA flow velocity above the threshold level. In this situation, the entire procedure may be done without modifying the state of flow. Adjustments may be made as needed if the MCA flow velocity changes during the course of the procedure, or the patient exhibits neurologic symptoms.

Exemplary Mechanisms to Regulate Flow

The system 100 is adapted to regulate retrograde flow in a variety of manners. Any combination of the pump 1110, valve 1115, syringe 1120, and/or variable resistance component 1125 can be manually controlled by the user or automatically controlled via the controller 1130 to adjust the retrograde flow rate. Thus, the system 100 can regulate retrograde flow in various manners, including controlling an active flow component (e.g., pump, syringe, etc.), reducing the flow restriction, switching to an aspiration source (such as a pre-set VacLock syringe, Vacutainer, suction system, or the like), or any combination thereof.

In the situation where an external receptacle or reservoir is used, the retrograde flow may be augmented in various manners. The reservoir has a head height comprised of the height of the blood inside the reservoir and the height of the reservoir with respect to the patient. Reverse flow into the reservoir may be modulated by setting the reservoir height to increase or decrease the amount of pressure gradient from the CCA to the reservoir. In an embodiment, the reservoir is raised to increase the reservoir pressure to a pressure that is greater than venous pressure. Or, the reservoir can be positioned below the patient, such as down to a level of the floor, to lower the reservoir pressure to a pressure below venous or atmospheric pressure.

The variable flow resistance in shunt 120 may be provided in a wide variety of ways. In this regard, flow resistance component 1125 can cause a change in the size or shape of the shunt to vary flow conditions and thereby vary the flow rate. Or, the flow resistance component 1125 can re-route the blood flow through one or more alternate flow pathways in the shunt to vary the flow conditions. Some exemplary embodiments of the flow resistance component 1125 are now described.

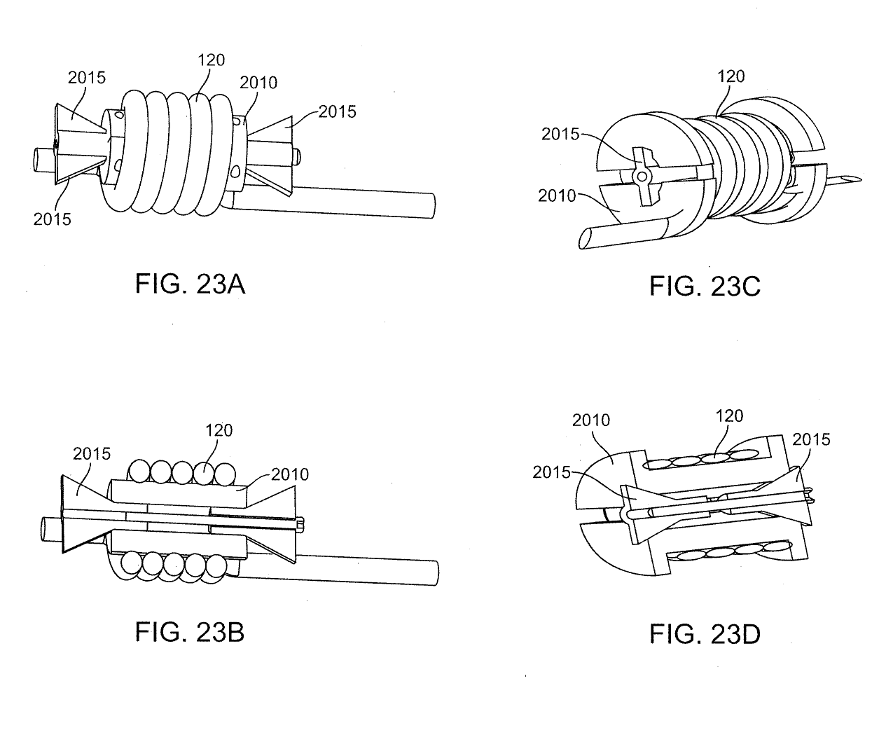

As shown in FIGS. 16A, 16B, 16C, and 16D, in an embodiment the shunt 120 has an inflatable bladder 1205 formed along a portion of its interior lumen. As shown in FIGS. 16A and 16C, when the bladder 1205 is deflated, the inner lumen of the shunt 120 remains substantially unrestricted, providing for a low resistance flow. By inflating the bladder 1205, however, as shown in FIGS. 16B and 16D, the flow lumen can be greatly restricted, thus greatly increasing the flow resistance and reducing the flow rate of atrial blood to the venous vasculature. The controller 1130 can control inflation/deflation of the bladder 1205 or it can be controlled manually by the user.

Rather than using an inflatable internal bladder, as shown in FIGS. 16A-16D, the cross-sectional area of the lumen in the shunt 120 may be decreased by applying an external force, such as flattening the shunt 120 with a pair of opposed plates 1405, as shown in FIGS. 17A-17D. The opposed plates are adapted to move toward and away from one another with the shunt 120 positioned between the plates. When the plates 1405 are spaced apart, as shown in FIGS. 17A and 17C, the lumen of the shunt 120 remains unrestricted. When the plates 1405 are closed on the shunt 120, as shown in FIGS. 17B and 17D, in contrast, the plates 1405 constrict the shunt 120. In this manner, the lumen remaining in shunt 120 can be greatly decreased to increase flow resistance through the shunt. The controller 1130 can control movement of the plates 1405 or such movement can be controlled manually by the user.

Referring now to FIGS. 18A and 18B, the available cross-sectional area of the shunt 120 can also be restricted by axially elongating a portion 1505 of the shunt 120. Prior to axial elongation, the portion 1505 will be generally unchanged, providing a full luminal flow area in the portion 1505, as shown in FIG. 18A. By elongating the portion 1505, however, as shown in FIG. 18B, the internal luminal area of the shunt 120 in the portion 1505 can be significantly decreased and the length increased, both of which have the effect of increasing the flow resistance. When employing axial elongation to reduce the luminal area of shunt 120, it will be advantageous to employ a mesh or braid structure in the shunt at least in the portion 1505. The mesh or braid structure provides the shunt 120 with a pliable feature that facilitates axial elongation without breaking. The controller 1130 can control elongation of the shunt 120 or such it can be controlled manually by the user.

Referring now to FIGS. 19A-19D, instead of applying an external force to reduce the cross-sectional area of shunt 120, a portion of the shunt 120 can be made with a small diameter to begin with, as shown in FIGS. 19A and 19C. The shunt 120 passes through a chamber 1600 which is sealed at both ends. A vacuum is applied within the chamber 1600 exterior of the shunt 120 to cause a pressure gradient. The pressure gradient cause the shunt 120 to increase in size within the chamber 120, as shown in FIGS. 16B and 12D. The vacuum may be applied in a receptacle 1605 attached to a vacuum source 1610. Conversely, a similar system may be employed with a shunt 120 whose resting configuration is in the increased size. Pressure may be applied to the chamber to shrink or flatten the shunt to decrease the flow resistance. The controller 1130 can control the vacuum or it can be controlled manually by the user.

As yet another alternative, the flow resistance through shunt 120 may be changed by providing two or more alternative flow paths. As shown in FIG. 20A, the flow through shunt 120 passes through a main lumen 1700 as well as secondary lumen 1705. The secondary lumen 1705 is longer and/or has a smaller diameter than the main lumen 1700. Thus, the secondary lumen 1705 has higher flow resistance than the main lumen 1700. By passing the blood through both these lumens, the flow resistance will be at a minimum. Blood is able to flow through both lumens 1700 and 1705 due to the pressure drop created in the main lumen 1700 across the inlet and outlet of the secondary lumen 1705. This has the benefit of preventing stagnant blood. As shown in FIG. 20B, by blocking flow through the main lumen 1700 of shunt 120, the flow can be diverted entirely to the secondary lumen 1705, thus increasing the flow resistance and reducing the blood flow rate. It will be appreciated that additional flow lumens could also be provided in parallel to allow for a three, four, or more discrete flow resistances. The shunt 120 may be equipped with a valve 1710 that controls flow to the main lumen 1700 and the secondary lumen 1705 with the valve 1710 being controlled by the controller 1130 or being controlled manually by the user. The embodiment of FIGS. 20A and 20B has an advantage in that this embodiment in that it does not require as small of lumen sizes to achieve desired retrograde flow rates as some of the other embodiments of variable flow resistance mechanisms. This is a benefit in blood flow lines in that there is less chance of clogging and causing clots in larger lumen sizes than smaller lumen sizes.

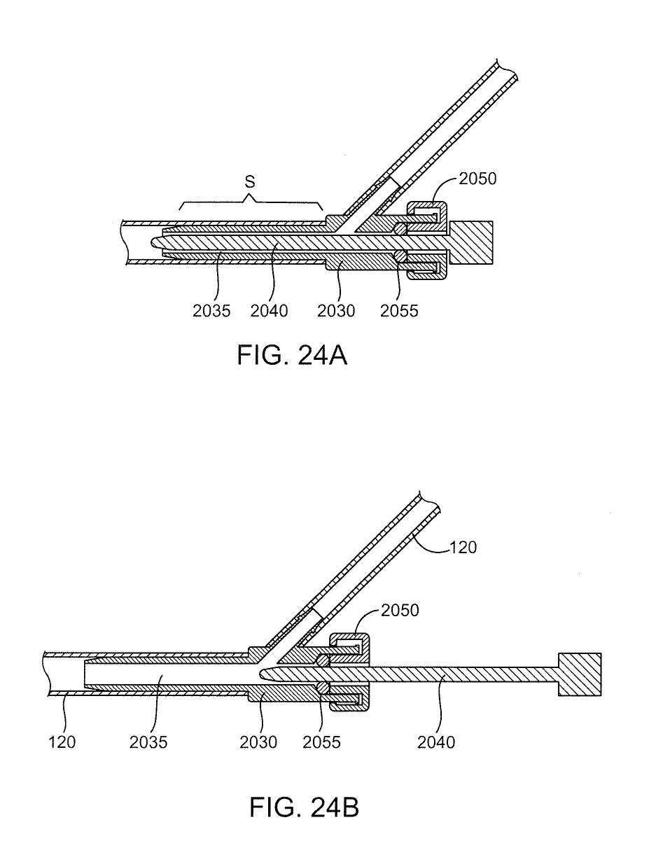

The shunt 120 can also be arranged in a variety of coiled configurations which permit external compression to vary the flow resistance in a variety of ways. Arrangement of a portion of the shunt 120 in a coil contains a long section of the shunt in a relatively small area. This allows compression of a long length of the shunt 120 over a small space. As shown in FIGS. 21A and 21B, a portion of the shunt 120 is wound around a dowel 1805 to form a coiled region. The dowel 1805 has plates 1810a and 1810b which can move toward and away from each other in an axial direction. When plates 1810a and 1810b are moved away from each other, the coiled portion of the shunt 105 is uncompressed and flow resistance is at a minimum. The shunt 120 is large diameter, so when the shunt is non-compressed, the flow resistance is low, allowing a high-flow state. To down-regulate the flow, the two plates 1810a and 1810b are pushed together, compressing the coil of shunt 120. By moving the plates 1810a and 1810b together, as shown in FIG. 21B, the coiled portion of the shunt 120 is compressed to increase the flow resistance. The controller 1130 can control the plates or they can be controlled manually by the user.

A similar compression apparatus is shown in FIGS. 22A and 22B. In this configuration, the coiled shunt 120 is encased between two movable cylinder halves 1905a and 1905b. The halves 1905a and 1905b can slide along dowel pins 1910 to move toward and away from one another. When the cylinder halves 1905 are moved apart, the coiled shunt 120 is uncompressed and flow resistance is at a minimum. When the cylinder halves 1905 are brought together, the coiled shunt 120 is compressed circumferentially to increase flow resistance. The controller 1130 can control the halves 1905 or they can be controlled manually by the user.

As shown in FIGS. 23A through 23D, the shunt 120 may also be wound around an axially split mandrel 2010 having wedge elements 2015 on opposed ends. By axially translating wedge elements 2015 in and out of the split mandrel 2010, the split portions of the mandrel are opened and closed relative to one another, causing the coil of tubing to be stretched (when the mandrel portions 2010 are spread apart, FIGS. 23C, 23D) or relaxed (when the mandrel portions 2010 are closed, FIGS. 23A, 23B.) Thus, when the wedge elements 2015 are spaced apart, as shown in FIGS. 23A and 23B, the outward pressure on the shunt 120 is at a minimum and the flow resistance is also at a minimum. By driving the wedge elements 2015 inwardly, as shown in FIGS. 23C and 23D, the split mandrel halves 2020 are forced apart and the coil of shunt 120 is stretched. This has the dual effect of decreasing the cross sectional area of the shunt and lengthening the shunt in the coiled region, both of which lead to increased flow resistance.

FIGS. 24A and 24B show an embodiment of the variable resistance component 1125 that uses a dowel to vary the resistance to flow. A housing 2030 is inserted into a section of the shunt 120. The housing 2030 has an internal lumen 2035 that is contiguous with the internal lumen of the shunt 120. A dowel 2040 can move into and out of a portion of the internal lumen 2035. As shown in FIG. 24A, when the dowel 2040 is inserted into the internal lumen 2035, the internal lumen 2035 is annular with a cross-sectional area that is much smaller than the cross-sectional area of the internal lumen 2035 when the dowel is not present. Thus, flow resistance increases when the dowel 2040 is positioned in the internal lumen 2035. The annular internal lumen 2035 has a length S that can be varied by varying the portion of the dowel 2040 that is inserted into the lumen 2035. Thus, as more of the dowel 2040 is inserted, the length S of the annular lumen 2035 increases and vice-versa. This can be used to vary the level of flow resistance caused by the presence of the dowel 2040.

The dowel 2040 enters the internal lumen 2035 via a hemostasis valve in the housing 2030. A cap 2050 and an O-ring 2055 provide a sealing engagement that seals the housing 2030 and dowel 2040 against leakage. The cap 2050 may have a locking feature, such as threads, that can be used to lock the cap 2050 against the housing 2030 and to also fix the position of the dowel 2040 in the housing 2040. When the cap 2050 is locked or tightened, the cap 2050 exerts pressure against the O-ring 2055 to tighten it against the dowel 2040 in a sealed engagement. When the cap 2050 is unlocked or untightened, the dowel 2040 is free to move in and out of the housing 2030.

A closing element (also referred to as a closure device), such as a self-closing element, may be deployed about the penetration in the wall of the common carotid artery prior to withdrawing the sheath 605 at the end of the procedure. The closure device may be deployed before placement of an arterial access sheath into the artery. Usually, the closing element will be deployed at or near the beginning of the procedure, but optionally, the closing element could be deployed as the sheath is being withdrawn, often being released from a distal end of the sheath onto the wall of the common carotid artery. Use of a self-closing element is advantageous since it affects substantially the rapid closure of the penetration in the artery as the sheath is being withdrawn. Such rapid closure can reduce or eliminate unintended blood loss either at the end of the procedure or during accidental dislodgement of the sheath. In addition, such a self-closing element may reduce the risk of arterial wall dissection during access. Further, the closing element may be configured to exert a frictional or other retention force on the sheath during the procedure. Such a retention force is advantageous and can reduce the chance of accidentally dislodging the sheath during the procedure. A self-closing element eliminates the need for vascular surgical closure of the artery with suture after sheath removal, reducing the need for a large surgical field and greatly reducing the surgical skill required for the procedure.

The disclosed systems and methods may employ a wide variety of closing elements, including self-closing elements, typically being mechanical elements which include an anchor portion and a closing or self-closing portion. The anchor portion may comprise hooks, pins, staples, clips, tine, suture, or the like, which are engaged in the exterior surface of the common carotid artery about the penetration to immobilize the self-closing element when the penetration is fully open. The closing element may also include a spring-like or other self-closing portion which, upon removal of the sheath, will close the anchor portion in order to draw the tissue in the arterial wall together to provide closure. Usually, the closure will be sufficient so that no further measures need be taken to close or seal the penetration. Optionally, however, it may be desirable to provide for supplemental sealing of the self-closing element after the sheath is withdrawn. For example, the self-closing element and/or the tissue tract in the region of the element can be treated with hemostatic materials, such as bioabsorbable polymers, collagen plugs, glues, sealants, clotting factors, or other clot-promoting agents. Alternatively, the tissue or self-closing element could be sealed using other sealing protocols, such as electrocautery, suturing, clipping, stapling, or the like. In another method, the self-closing element will be a self-sealing membrane or gasket material which is attached to the outer wall of the vessel with clips, glue, bands, or other means. The self-sealing membrane may have an inner opening such as a slit or cross cut, which would be normally closed against blood pressure. Any of these self-closing elements could be designed to be placed in an open surgical procedure, or deployed percutaneously.

In an embodiment, the closing element is a is a suture-based blood vessel closure device that can perform the dilation of an arteriotomy puncture, and therefore does not require previous dilation of the arteriotomy puncture by a separate device or by a procedural sheath dilator. The suture-based vessel closure device can place one or more sutures across a vessel access site such that, when the suture ends are tied off after sheath removal, the stitch or stitches provide hemostasis to the access site. The sutures can be applied either prior to insertion of a procedural sheath through the arteriotomy or after removal of the sheath from the arteriotomy. The device can maintain temporary hemostasis of the arteriotomy after placement of sutures but before and during placement of a procedural sheath and can also maintain temporary hemostasis after withdrawal of the procedural sheath but before tying off the suture. Some exemplary suture-based blood vessel disclosure devices are described in the following U.S. patents and patent applications, which are incorporated herein by reference in their entirety: U.S. Pat. No. 7,001,400 and U.S. Pat. No. 7,004,952.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A system of devices for treating cerebral artery disease or the brain, comprising:
an arterial access sheath adapted to be introduced into a common carotid, internal carotid, or vertebral artery through a penetration in the artery and receive blood from the artery;
a shunt fluidly connected to the arterial access sheath, wherein the shunt provides a pathway for blood to flow from the arterial access sheath to a return site;
a flow control assembly coupled to the shunt and adapted to regulate blood flow through the shunt; and
a treatment device adapted to be introduced into the artery through the arterial access sheath and configured to treat the cerebral artery or brain.

2. A system of devices as in claim 1, further comprising an aspiration source connected to the shunt and adapted to aspirate the artery.

3. A system of devices as in claim 1, wherein the arterial access sheath has a step or taper to a reduced diameter at a distal end of the arterial access sheath to allow insertion of the distal end into a common carotid or vertebral artery while maintaining a larger diameter in the section of sheath outside the artery.

4. A system of devices as in claim 3, wherein the reduced diameter section is between 3 and 5 cm in length.

5. A system of devices as in claim 1, wherein the arterial access sheath has a step or taper to a reduced diameter at the distal end to allow insertion of the distal end into an internal carotid artery, distal vertebral or basilar artery.

6. A system of devices as in claim 5, wherein the reduced diameter section is between 10 and 15 cm in length.

7. A system of devices as in claim 1, further comprising an occlusion element on the distal end of the arterial access sheath.

8. A system of devices as in claim 7, wherein the occlusion element is a balloon.

9. A system of devices as in claim 1, further comprising a closure device adapted to provide a closure force to the penetration.

10. A system of devices as in claim 9, wherein the closure device is a suture-based closure device.

11. A system of devices as in claim 9, wherein the closure device is a self-closing clip.

12. A system of devices as in claim 1, wherein the treatment device comprises an embolic system which delivers an embolic coil, material or fluid composition.

13. A system of devices as in claim 1, wherein the treatment device comprises a stent delivery catheter.

14. A system of devices as in claim 1, wherein the treatment device comprises balloon dilatation catheter.

15. A system of devices as in claim 1, wherein the treatment device comprises a balloon occlusion catheter.

16. A system of devices as in claim 1, wherein the treatment device comprises a microcatheter that delivers a therapeutic agent.

17. A system of devices as in claim 16, wherein the therapeutic agent comprises at least one of a cerebral protective agent, a chemotherapeutic agent, a stem cell, a regenerative agents, a neurochemical agent, or neuropsychopharmacologic agent.

18. A system of devices as in claim 1, wherein the treatment device comprises a thrombus removal system.

19. A system of devices as in claim 1, wherein the treatment device comprises a diagnostic angiography catheter.

20. A system of devices as in claim 1, wherein the treatment device comprises a brain tumor treatment device.

21. A system of devices for treating cerebral artery disease or the brain, comprising:
an arterial access sheath adapted to be introduced into a common carotid, internal carotid, or vertebral artery through a penetration in the artery and receive blood from the artery;
a treatment device adapted to be introduced into the artery through the arterial access sheath and configured to treat the cerebral artery or the brain; and
a closure device adapted to provide a closure force to the penetration.

22. A system of devices as in claim 21, wherein the closure device is a suture-based closure device.

23. A system of devices as in claim 21, wherein the closure device is a self-closing clip.

24. A system of devices as in claim 21, wherein the closure device is configured to be applied prior to insertion of the sheath.

25. A system of devices as in claim 21, wherein the treatment device comprises an embolic system which delivers an embolic coil, material or fluid composition.

26. A system of devices as in claim 21, wherein the treatment device comprises a stent delivery catheter.

27. A system of devices as in claim 21, wherein the treatment device comprises balloon dilatation catheter.

28. A system of devices as in claim 21, wherein the treatment device comprises a balloon occlusion catheter.

29. A system of devices as in claim 21, wherein the treatment device comprises a microcatheter that delivers a therapeutic agent.

30. A system of devices as in claim 29, wherein the therapeutic agent comprises at least one of a cerebral protective agent, a chemotherapeutic agent, a stem cell, a regenerative agents, a neurochemical agent, or neuropsychopharmacologic agent.

31. A system of devices as in claim 21, wherein the treatment device comprises a thrombus removal system.

32. A system of devices as in claim 21, wherein the treatment device comprises a diagnostic angiography catheter.

33. A system of devices as in claim 21, wherein the treatment device comprises a brain tumor treatment device.

34. A method for treatment of cerebral artery disease or the brain, comprising:
forming a penetration in a wall of a common carotid, internal carotid, or vertebral artery;
positioning an arterial access sheath through the penetration into the artery;
occluding at least one of the common or internal carotid artery or vertebral or basilar artery; and
treating the cerebral artery or the brain using a treatment device.

35. A method as in claim 34, further comprising establishing retrograde blood flow through the artery such that blood flows into a lumen of the arterial access sheath and into a shunt connected to the access sheath.

36. A method as in claim 35, further comprising causing blood to flow from the shunt to venous sheath and into a venous return site.

37. A method as in claim 34, further comprising causing blood to flow from the shunt to an external receptacle.

38. A method as in claim 34, further comprising regulating the retrograde blood flow.

39. A method as in claim 34, further comprising controlling a state of the retrograde blood flow.

40. A method as in claim 34, further comprising monitoring the retrograde blood flow.

41. A method as in claim 34, wherein the treatment device comprises an embolic system which delivers an embolic coil, material or fluid composition.

42. A method as in claim 34, wherein the treatment device comprises a stent delivery catheter.

43. A method as in claim 34, wherein the treatment device comprises balloon dilatation catheter.

44. A method as in claim 34, wherein the treatment device comprises a balloon occlusion catheter.

45. A method as in claim 34, wherein the treatment device comprises a microcatheter that delivers a therapeutic agent.

46. A method as in claim 45, wherein the therapeutic agent comprises at least one of a cerebral protective agent, a chemotherapeutic agent, a stem cell, a regenerative agents, a neurochemical agent, or neuropsychopharmacologic agent.

47. A method as in claim 34, wherein the treatment device comprises a thrombus removal system.

48. A method as in claim 34, wherein the treatment device comprises a diagnostic angiography catheter.

49. A method as in claim 34, wherein the treatment device comprises a brain tumor treatment device.

50. A method as in claim 34, wherein the means for occlusion is an occlusion element attached to the arterial access sheath.

51. A method as in claim 50, wherein the occlusion element is a balloon

52. A method as in claim 50, wherein the occlusion element is a mechanical occlusion.

53. A method as in claim 34, further comprising actively aspirating through the arterial access device.

54. A method as in claim 53, wherein the aspiration means comprises a syringe.

55. A method as in claim 53, wherein the aspiration means comprises a suction pump and collection reservoir.

56. A method for treatment of cerebral artery disease or the brain, comprising:
forming a percutaneous penetration in a wall of a common carotid, internal carotid, or vertebral artery;
applying a closure device at a site of penetration before placement of an arterial access sheath;
positioning an arterial access sheath through the penetration into the artery;
treating the cerebral artery or the brain using a treatment device; and
closing the access site with the closure device.

57. A method as in claim 56, wherein the closure device comprises a suture-based closure device.

58. A method as in claim 56, wherein the closure device is a self-closing clip.

59. A method as in claim 56, wherein the treatment device comprises an embolic system which delivers an embolic coil, material or fluid composition.

60. A method as in claim 56, wherein the treatment device comprises a stent delivery catheter.

61. A method as in claim 56, wherein the treatment device comprises balloon dilatation catheter.

62. A method as in claim 56, wherein the treatment device comprises a balloon occlusion catheter.

63. A method as in claim 56, wherein the treatment device comprises a microcatheter that delivers a therapeutic agent.

64. A method as in claim 63, wherein the therapeutic agent comprises at least one of a cerebral protective agent, a chemotherapeutic agent, a stem cell, a regenerative agents, a neurochemical agent, or neuropsychopharmacologic agent.

65. A method as in claim 56, wherein the treatment device comprises a thrombus removal system.

66. A method as in claim 56, wherein the treatment device comprises a diagnostic angiography catheter.

67. A method as in claim 56, wherein the treatment device comprises a brain tumor treatment device.

68. A method as in claim 56, further comprising occluding at least one of the common or internal carotid artery or vertebral or basilar artery after positioning the arterial access site through the arterial penetration.

69. A method as in claim 68, further comprising establishing retrograde blood flow through the artery such that blood flows into a lumen of the arterial access sheath and into a shunt connected to the access sheath

70. A method as in claim 69, wherein the treatment device comprises an embolic system which delivers an embolic coil, material or fluid composition.

71. A method as in claim 69, wherein the treatment device comprises a stent delivery catheter.

72. A method as in claim 69, wherein the treatment device comprises balloon dilatation catheter.

73. A method as in claim 69, wherein the treatment device comprises a balloon occlusion catheter.

74. A method as in claim 69, wherein the treatment device comprises a microcatheter that delivers a therapeutic agent.

75. A method as in claim 74, wherein the therapeutic agent comprises at least one of a cerebral protective agent, a chemotherapeutic agent, a stem cell, a regenerative agents, a neurochemical agent, or neuropsychopharmacologic agent.

76. A method as in claim 69, wherein the treatment device comprises a thrombus removal system.

77. A method as in claim 69, wherein the treatment device comprises a diagnostic angiography catheter.

78. A method as in claim 69, wherein the treatment device comprises a brain tumor treatment device.

79. A method for treatment of cerebral artery disease or the brain, comprising:
forming a penetration in a wall of a common carotid, internal carotid or vertebral artery;
positioning an arterial access sheath through the penetration;
inserting a treatment device through the arterial access sheath into the common carotid or vertebral artery;
positioning at least a portion of the treatment device in the cerebral artery;
treating the cerebral artery or the brain using the treatment device.

80. A method as in claim 79, further comprising establishing an embolic protection condition.

81. A method as in claim 80, wherein establishing an embolic protection condition comprises deploying an occlusion element in the common or internal carotid artery or vertebral artery to block the artery.

82. A method as in claim 81, wherein the occlusion element is a balloon.

83. A method as in claim 81, wherein the occlusion element is a mechanical occlusion.

84. A method as in claim 81, wherein the occlusion element is attached to the arterial access sheath.

85. A method as in claim 81, wherein establishing an embolic protection condition further comprises actively aspirating the artery through the arterial access sheath.

86. A method as in claim 81, wherein the arterial access sheath includes a connection to a shunt and establishing an embolic protection condition further comprises establishing retrograde blood flow through the common carotid or vertebral artery such that blood flows into a lumen of the arterial access sheath and into the shunt.

87. A method as in claim 86, further comprising causing blood to flow from the shunt to venous sheath and into a venous return site.

88. A method as in claim 86, further comprising causing blood to flow from the shunt to an external receptacle.

89. A method as in claim 86, further comprising regulating the retrograde blood flow.

90. A method as in claim 86, further comprising controlling a state of the retrograde blood flow.

91. A method as in claim 86, further comprising monitoring the retrograde blood flow.

92. A method as in claim 79, wherein the penetration is formed percutaneously.

93. A method as in claim 92, further comprising applying a closure device to the penetration in the common carotid or vertebral artery to close the penetration.

94. A method as in claim 93, wherein the closure device comprises a suture-based device.

95. A method as in claim 93, wherein the closure device is applied prior to positioning an arterial access sheath through the penetration.

96. A method as in claim 93, wherein the closure device is a self-closing clip.

97. A method as in claim 79, further comprising identifying an indication associated with cerebral artery or cerebral disease.

98. A method as in claim 79, wherein the treatment device comprises an embolic system which delivers an embolic coil, material or fluid composition.

99. A method as in claim 79, wherein the treatment device comprises a stent delivery catheter.

100. A method as in claim 79, wherein the treatment device comprises balloon dilatation catheter.

101. A method as in claim 79, wherein the treatment device comprises a balloon occlusion catheter.

102. A method as in claim 79, wherein the treatment device comprises a microcatheter that delivers a therapeutic agent.

103. A method as in claim 102, wherein the therapeutic agent comprises at least one of a cerebral protective agent, a chemotherapeutic agent, a stem cell, a regenerative agents, a neurochemical agent, or neuropsychopharmacologic agent.

104. A method as in claim 79, wherein the treatment device comprises a thrombus removal system.

105. A method as in claim 79, wherein the treatment device comprises a diagnostic angiography catheter.

106. A method as in claim 79, wherein the treatment device comprises a brain tumor treatment device.
